(19) United States
(12) Patent Application Publication
(10) Pub. No.: US 2005/0216043 A1
(43) Pub. Date: Sep. 29, 2005

(54) Stented end graft vessel device for anastomosis and related methods for percutaneous placement

(76) Inventors: Duane D. Blatter, Salt Lake City, UT (US); Troy J. Orr, Draper, UT (US); Michael C. Barrus, Vienne, VA (US)

Correspondence Address: STOEL RIVES LLP - SLC, 201 SOUTH MAIN STREET, ONE UTAH CENTER, SALT LAKE CITY, UT, 84111, US

(21) Appl. No.: 10/811,079

(22) Filed: Mar. 26, 2004

(51) Int. Cl. A61B 17/08

(52) U.S. Cl. 606/153

(57) ABSTRACT

Fluid communication between vessels is obtained by joining a vessel with a graft vessel in an end-to-side anastomosis at one end of the graft vessel and by joining another vessel in a side-to-side anastomosis at the other end of the graft vessel. A compression ring device and a concentrically configured operator enable the end-to-side anastomosis to be achieved in an interdigitated configuration while controlling blood flow. A stent enables the side-to-side anastomosis to be achieved.

### TECHNICAL FIELD

The present invention relates generally to the field of anastomosis. More specifically, the present invention relates to methods, systems and devices for joining vessels together.

### BRIEF DESCRIPTION OF THE DRAWINGS

Understanding that drawings depict only typical embodiments of the invention and are not therefore to be considered to be limiting of its scope, the invention will be described and explained with additional specificity and detail through the use of the accompanying drawings. The drawings are listed below.

FIG. 1 is a perspective view of an embodiment of the graft vessel device positioned within a patient's body.

FIG. 2A is a perspective view of a patient receiving a catheter and having a guide wire directed to a remote anastomosis site.

FIG. 2B is an enlarged partial cross-sectional view of a vessel with a guide wire positioned in the lumen thereof.

FIG. 2C is an enlarged partial cross-sectional view like that of FIG. 2B depicting a guide wire loop snare extending from the guide wire catheter.

FIG. 2D is an enlarged partial cross-sectional view like that of FIG. 2C depicting another catheter inserted into the vessel with a wire extending toward the loop snare.

FIG. 2E is an enlarged partial cross-sectional view like that of FIG. 2D depicting a loop snare after snaring a wire.

FIG. 2F is an enlarged partial cross-sectional view like that of FIG. 2E depicting the catheter being retracted into the other catheter.

FIG. 2G is an enlarged partial cross-sectional view like that of FIG. 2F following retraction of the large catheter.

FIG. 2H is an enlarged partial cross-sectional view like that of FIG. 2G following introduction of an anvil pull.

FIG. 2I is an enlarged partial cross-sectional view like that of FIG. 2H depicting the anvil being abutted against the vessel wall.

FIG. 2I is an enlarged partial cross-sectional view like that of FIG. 2I depicting the engaging surface of the anvil being pulled against the vessel wall.

FIG. 3A is an enlarged perspective view of an embodiment of the graft vessel device.

FIG. 3B is an enlarged perspective view of a pair of anastomosis rings adapted for use with the graft vessel device.

FIG. 4A is a perspective view of compression driving components of an operator for compressing a ring device to anastomose the end of a graft vessel device in an end-to-side anastomosis with a target vessel.

FIG. 4B is an exploded perspective view of the components shown in FIG. 4A.

FIG. 5A is a cross-sectional view of an anvil and an operator positioned at an anastomosis site in preparation for an end-to-side anastomosis of a graft vessel device to a target vessel.

FIG. 5B is a cross-sectional view like that of FIG. 5A as the anvil is distending the vessel wall at the anastomosis site and is being pulled into the ring device.

FIG. 5C is a cross-sectional view like that of FIG. 5B following the formation of an opening in the vessel wall.

FIG. 5D is a cross-sectional view like that of FIG. 5C as the two anastomosis rings are being approximated.

FIG. 5E is a cross-sectional view like that of FIG. 5D following approximation of the two anastomosis rings.

FIG. 5F is a cross-sectional view like that of FIG. 5E as the ring device is pushed from the operator.

FIG. 5G is an enlarged cross-sectional view of the end-to-side anastomosis side of a graft vessel device following anastomosis and retraction of the anvil.

FIG. 6A is a perspective view of an operator performing an anastomosis.

FIG. 6B is a perspective view following detachment of the cutting assembly of the operator from the compression assembly of the operator.

FIG. 6C is a perspective view of the graft vessel device following anastomosis of the end-to-side portion.

FIG. 7A is a perspective view of the operator.

FIG. 7B is an exploded perspective view of the operator.

FIG. 7C is a perspective view of the proximal end of the operator.

FIG. 7D is a cross-sectional view of the portion of the operator which cannot be seen in FIGS. 5A-5F including the cutting assembly of the operator and the components of the operator which enable the approximator to drive the compression of the ring device. The entire compression assembly of the operator is not shown.

FIG. 7E is a cross-sectional view of just the operator cutting assembly of the operator with the safety interlock extension engaging the actuator knob.

FIG. 7F is a cross-sectional view like that of FIG. 7E following abutment of the anvil against the cutter to allow approximation to be initiated.

FIG. 8A is a perspective view of an introducer and tear-away sheath positioned within a vein and a graft vessel device anastomosed to an artery.

FIG. 8B is a perspective view like that of FIG. 8A depicting the stented end of the graft vessel device being introduced into a tear-away sheath.

FIG. 8C is a cross-sectional view of the stented end of the graft vessel device inside the tear-away sheath.

FIG. 8D is a perspective view of the stented end of the graft vessel device being introduced into the vein.

FIG. 8E is a perspective view like that of FIG. 8D depicting a tear-away sheath being torn away.

FIG. 8F is a perspective view following complete anastomosis of both ends of the graft vessel device.

FIG. 9A is a side view of the stented end of a graft vessel device with a coating.

FIG. 9B is a side view of the stented end of a graft vessel device with a segmented coating.

FIG. 9C is a side view of the stented end of a graft vessel device with an inwardly tapered end.

FIG. 9D is a side view of the stented end of a graft vessel device with an outwardly tapered end.

### DETAILED DESCRIPTION OF PREFERRED EMBODIMENTS

The invention described hereinafter relates to methods, systems and devices for forming an anastomosis. An anastomosis is an operative union of two hollow or tubular structures. Anastomotic structures can be part of a variety of systems, such as the vascular system, the digestive system or the genitourinary system. The operative union of two hollow or tubular structures enable the flow of uninterrupted flow through such structures.

An anastomosis is termed end-to-end when the terminal portions of tubular structures are anastomosed, and it is termed end-to-side when the terminal portion of a tubular structure is anastomosed to a lateral portion of another tubular or hollow structure. In an end-to-side anastomosis, the structure whose end is anastomosed is often referred to as the “graft vessel” while the structure whose side wall is anastomosed is referred to as the “receiving structure” or “target vessel.” The terms first vessel and second vessel are also used below and may refer to either a graft vessel or a target vessel. The anastomosis device may also be used to anastomose a catheter directly to a target vessel, without the use of a graft vessel.

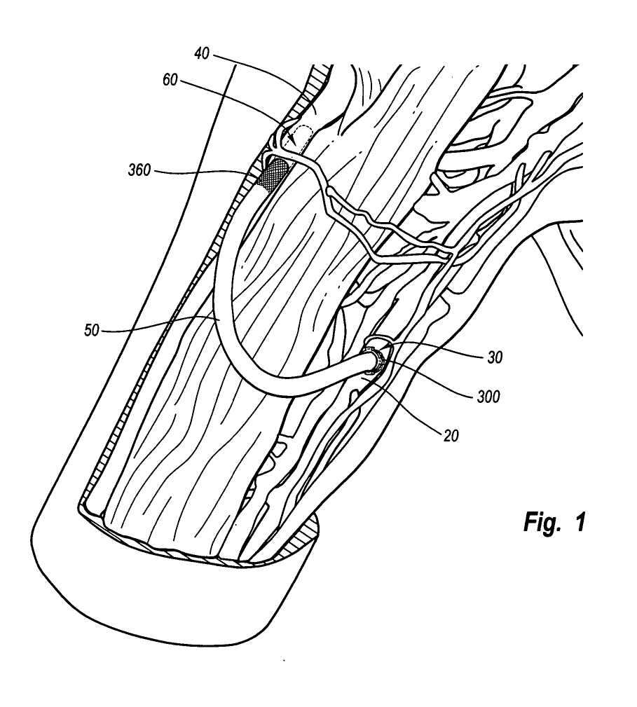

FIG. 1 depicts a graft vessel 50 anastomosed into a patient's arm. Such a configuration permits the graft vessel to be repeatedly accessed for procedures such as dialysis treatment. Graft vessel 50 is attached to an artery 20 at its anastomosis end 30 via an anastomosis ring device 300 in an end-to-side anastomosis. Graft vessel 50 is attached to a vein 40 at its stented end 60 via a stent 360 attached to the outside of graft vessel 50. Stented end 60 is attached to the vein in an end-to-end anastomosis.

Anastomosis necessarily requires a degree of invasion. The invasive character of an anastomosis, however, is minimized via the systems, methods and devices disclosed herein. The procedure involves several phases.

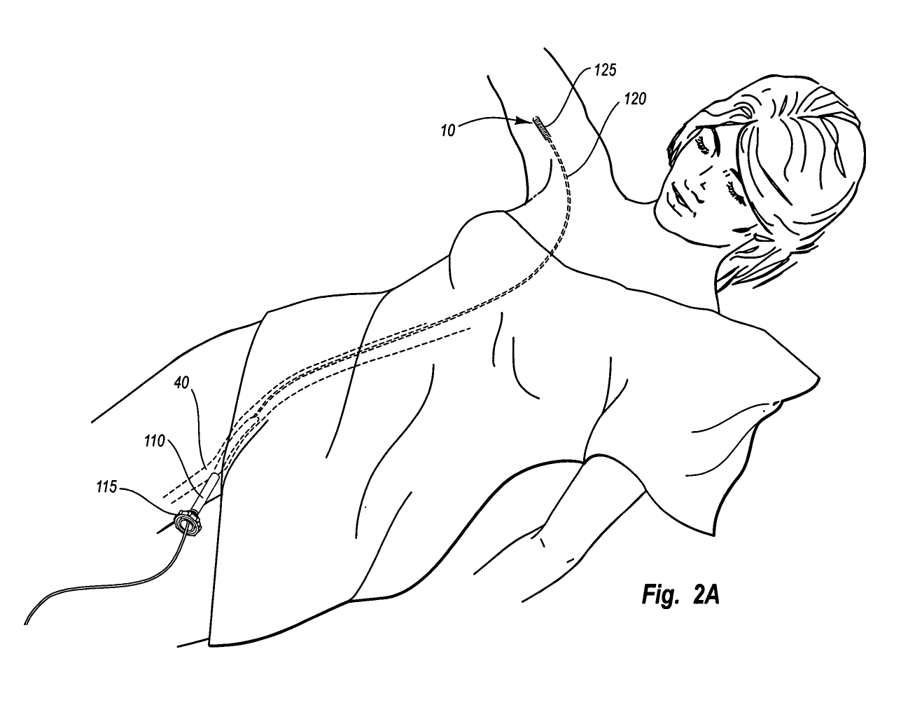

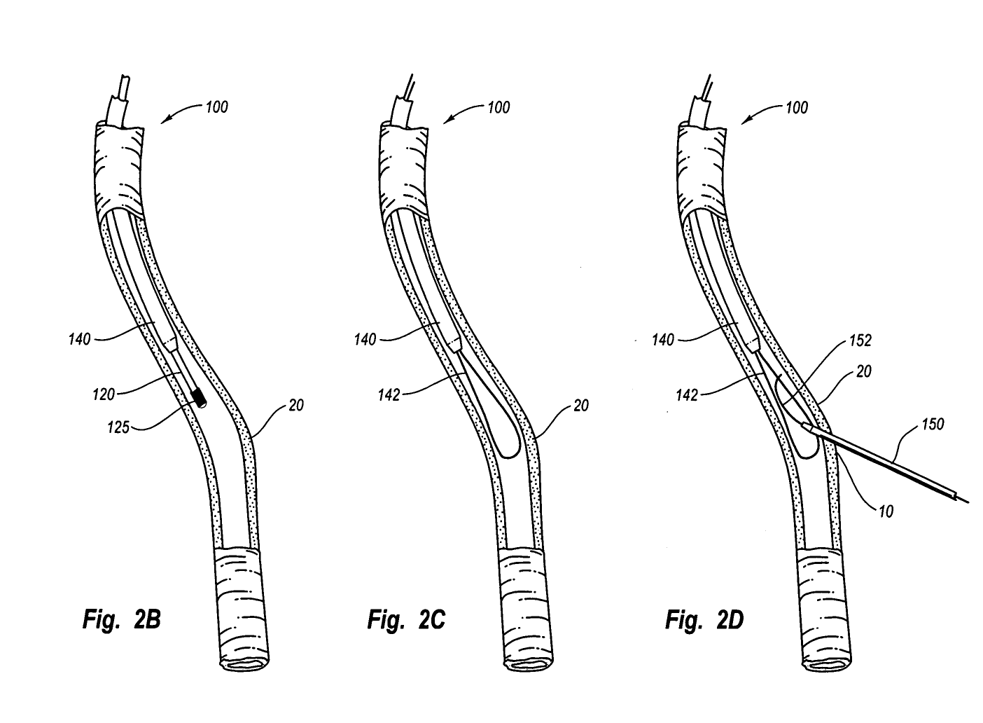

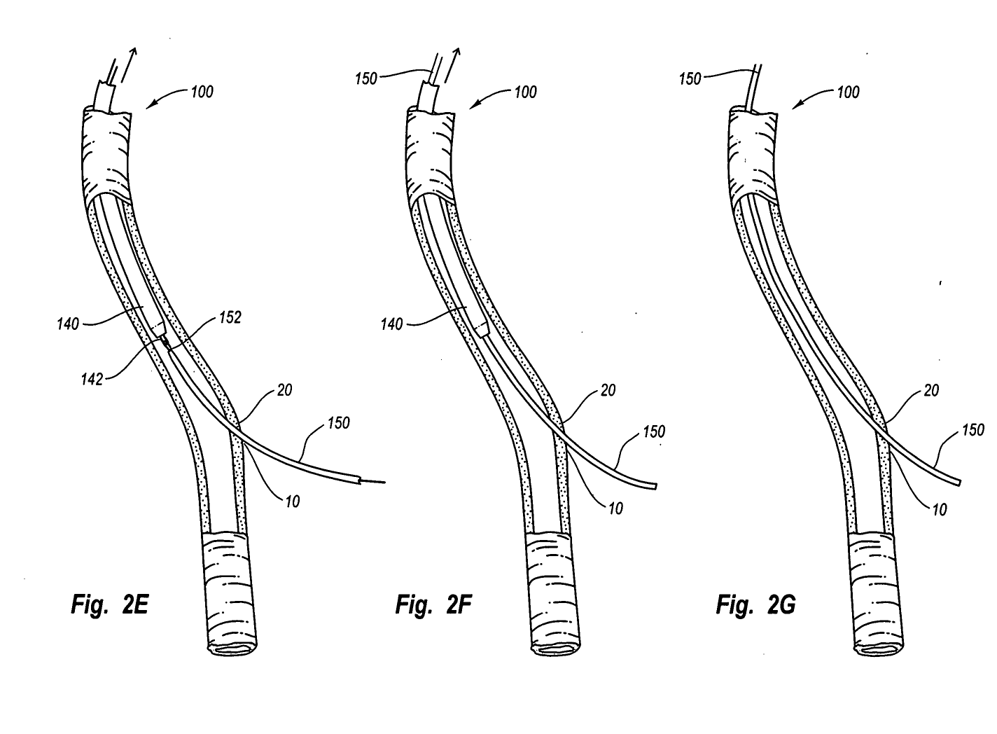

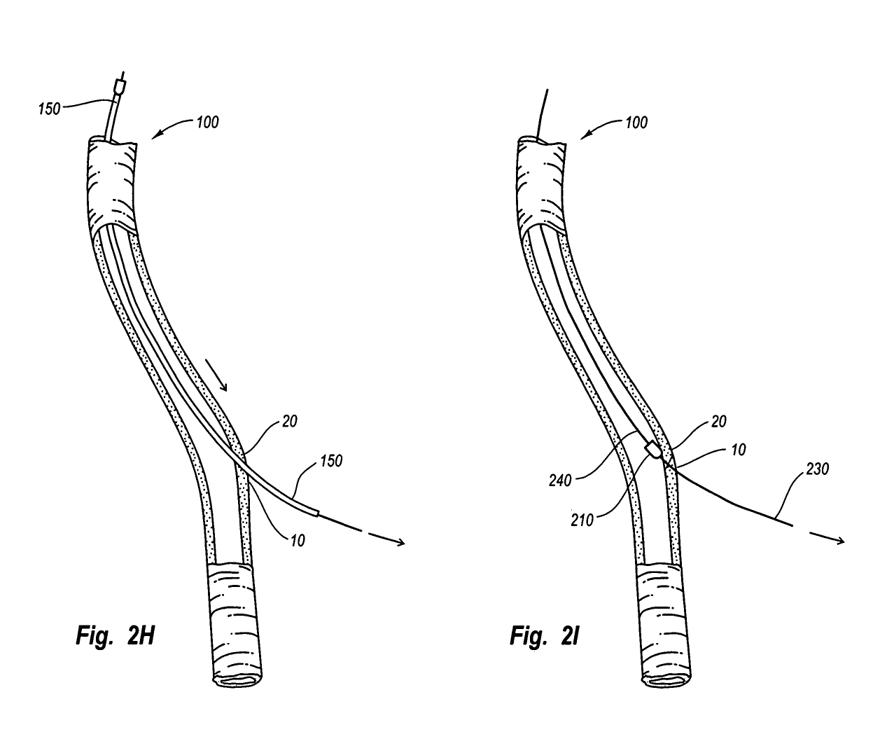

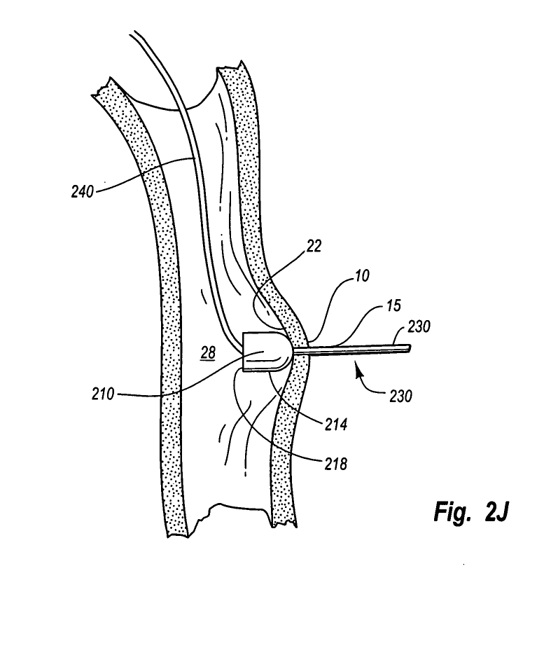

First an anvil apparatus 200 having an anvil pull 230 extending from an anvil 210 is positioned at an anastomosis site with anvil 210 in the lumen 28 of the target vessel 20 and its anvil pull extending beyond the vessel as shown in FIG. 2J. FIGS. 2A-2I depict an endovascular procedure for positioning an anvil as shown in FIG. 2J.

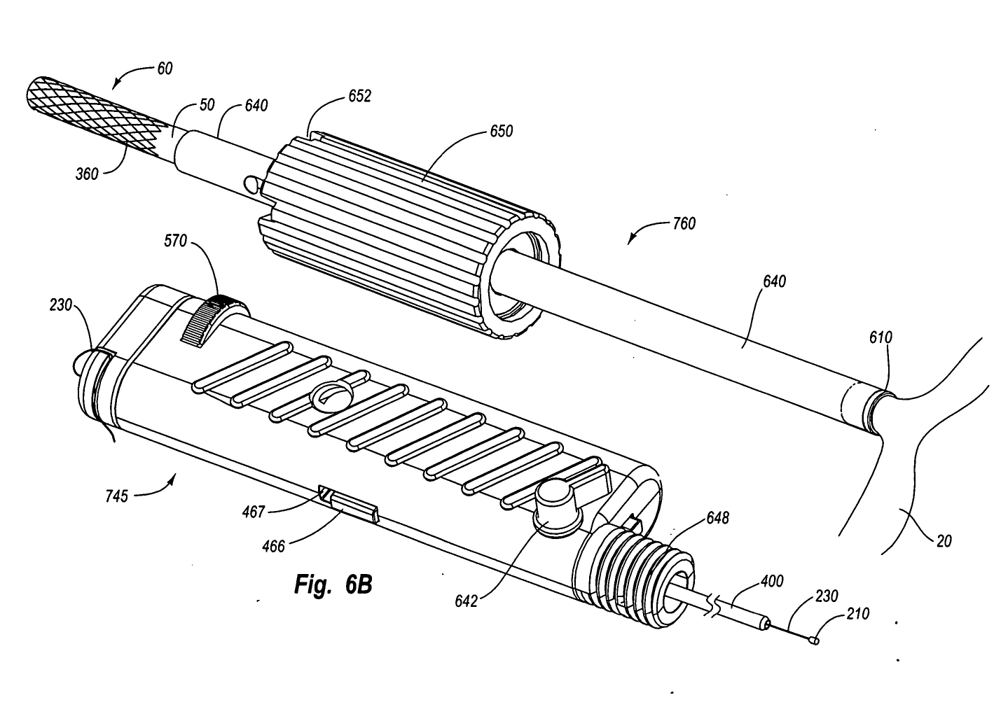

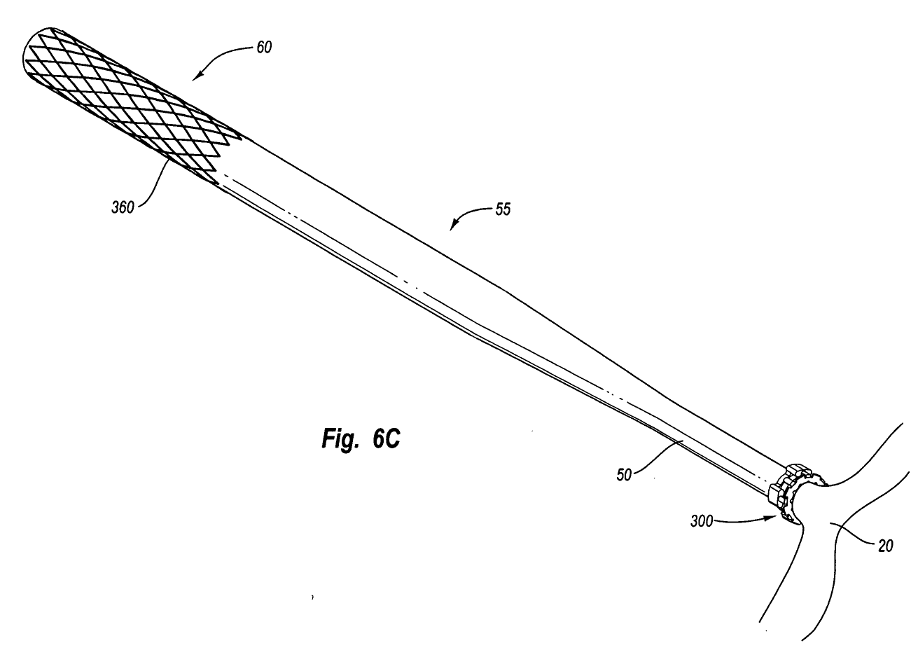

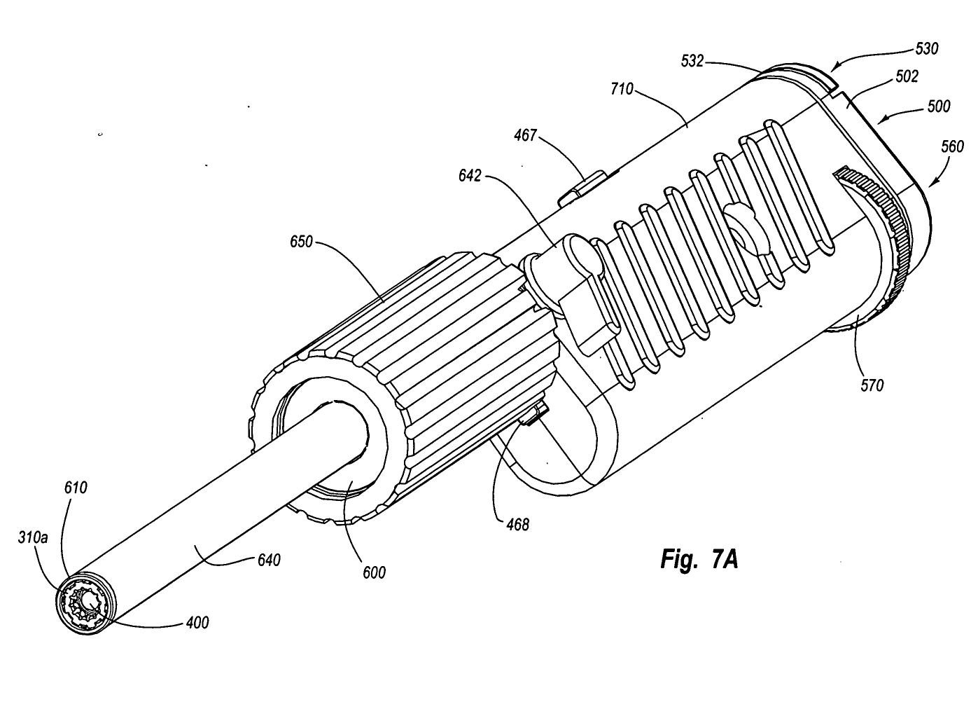

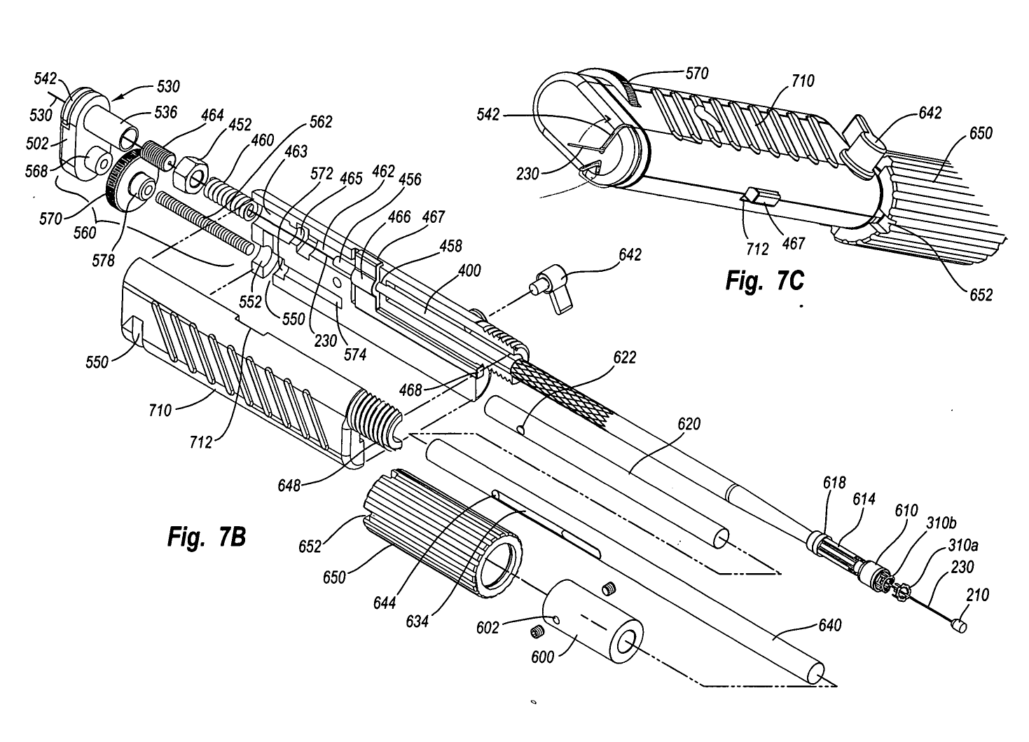

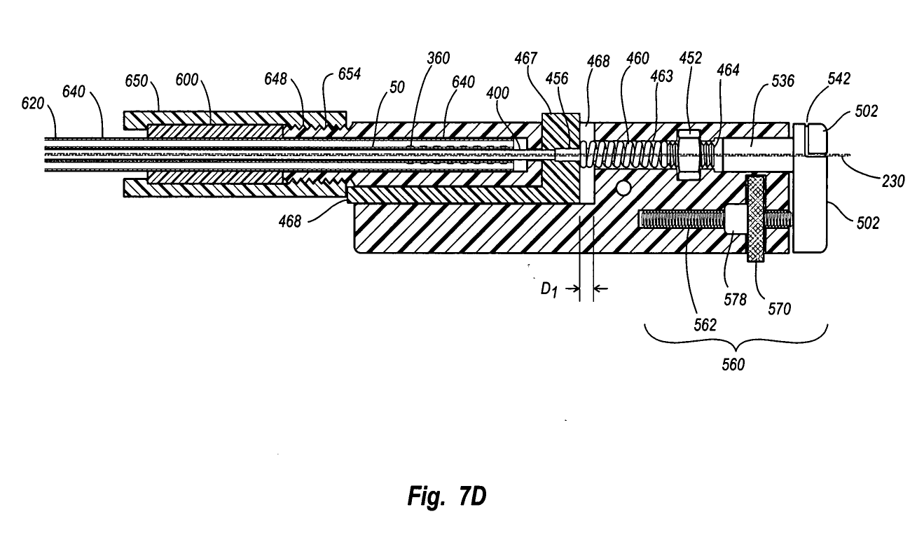

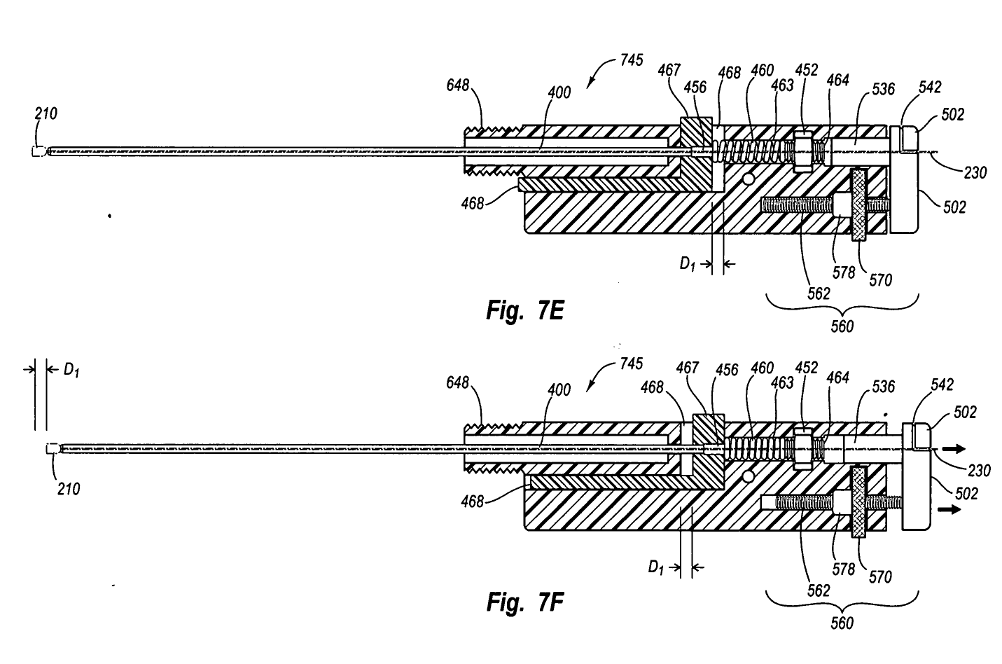

As shown in FIG. 6A, anvil pull 230 is then threaded concentrically through an external anastomosis operator or applicator 700 referred to herein as operator 700 which is positioned externally relative to target vessel 20. Once anvil pull 230 is coupled to operator 700 then an anastomosis fenestra or opening is formed in the target vessel. The specific components used to create the opening include anvil apparatus 200 and cutting assembly 745 of operator 700 shown in FIGS. 7E-7F. Note that cutting assembly 745 of operator 700 is shown in FIGS. 7E-7F detached from compression assembly 760 just for illustrative purposes. Cutting assembly 745 and compression assembly 760 are not separated as shown in FIG. 6B until after the end-to-side anastomosis is completed.

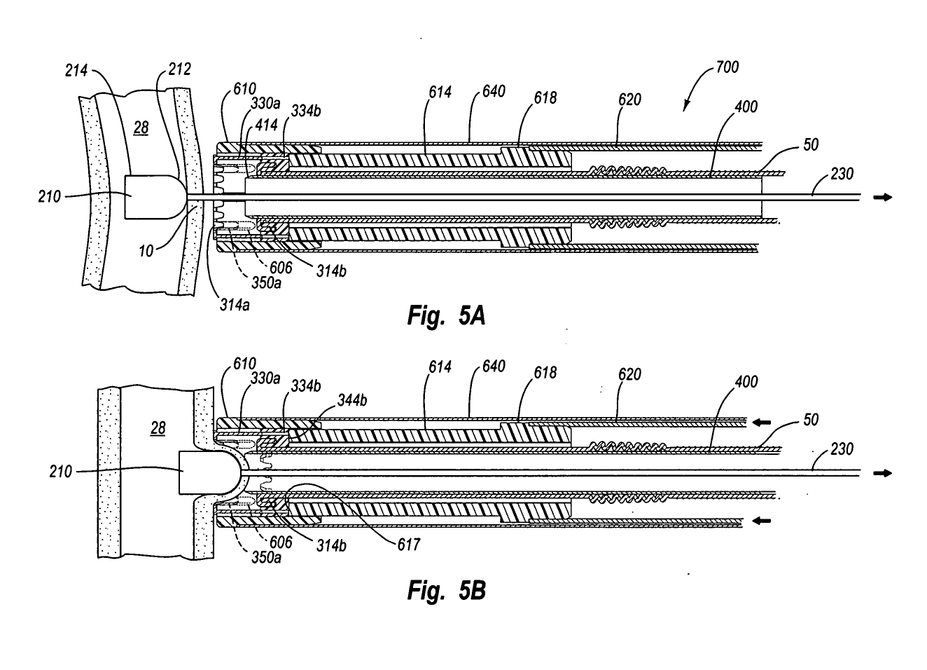

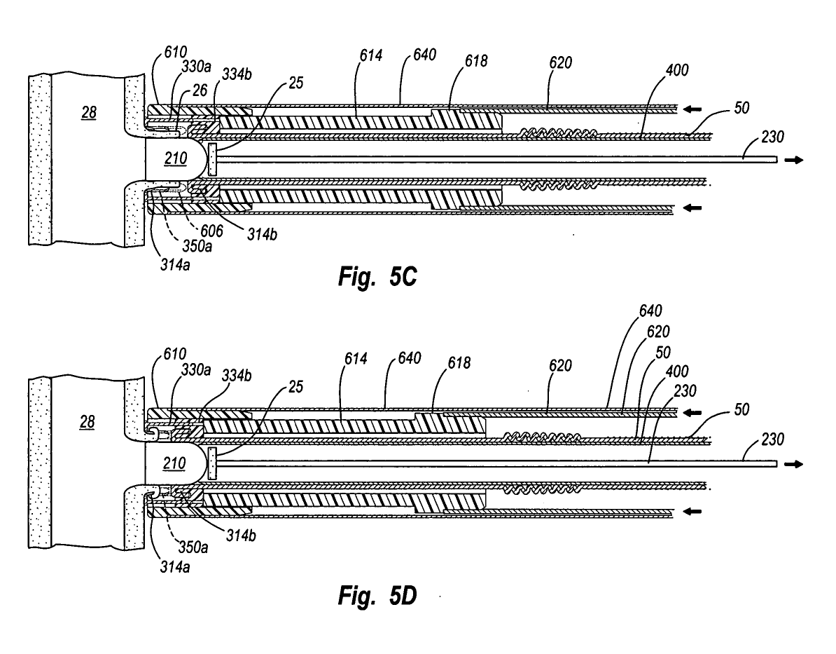

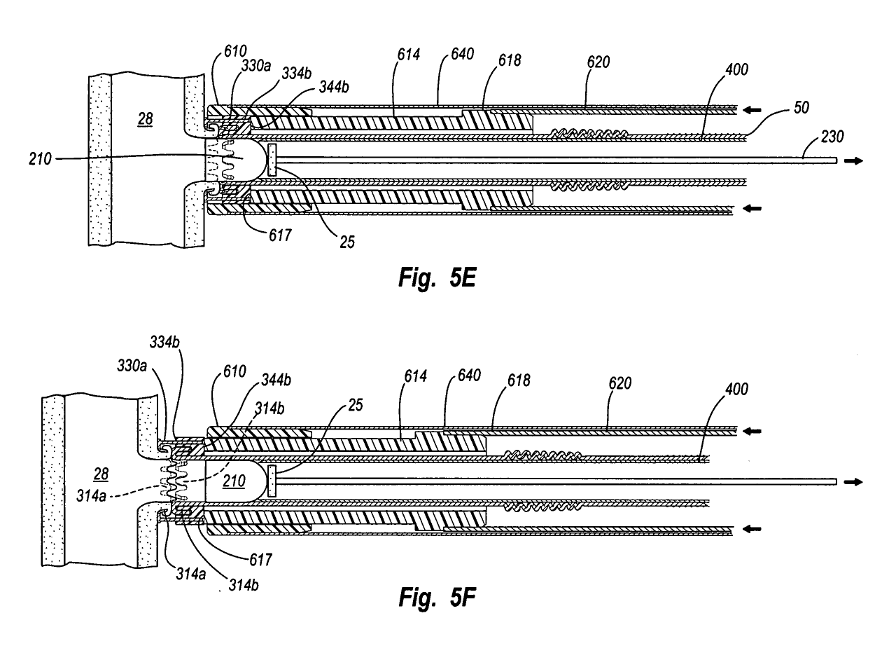

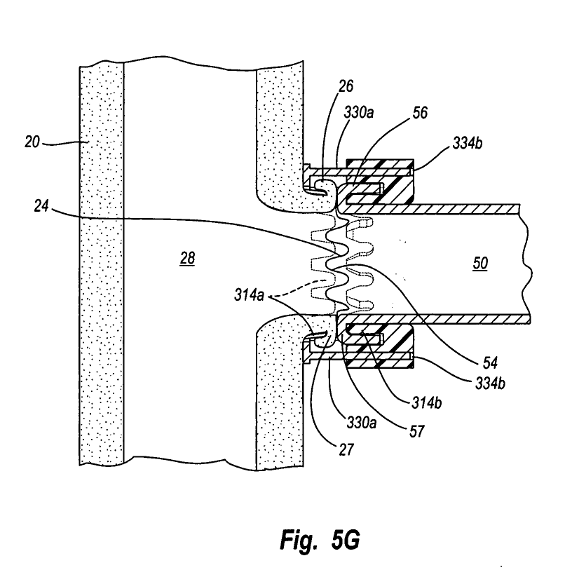

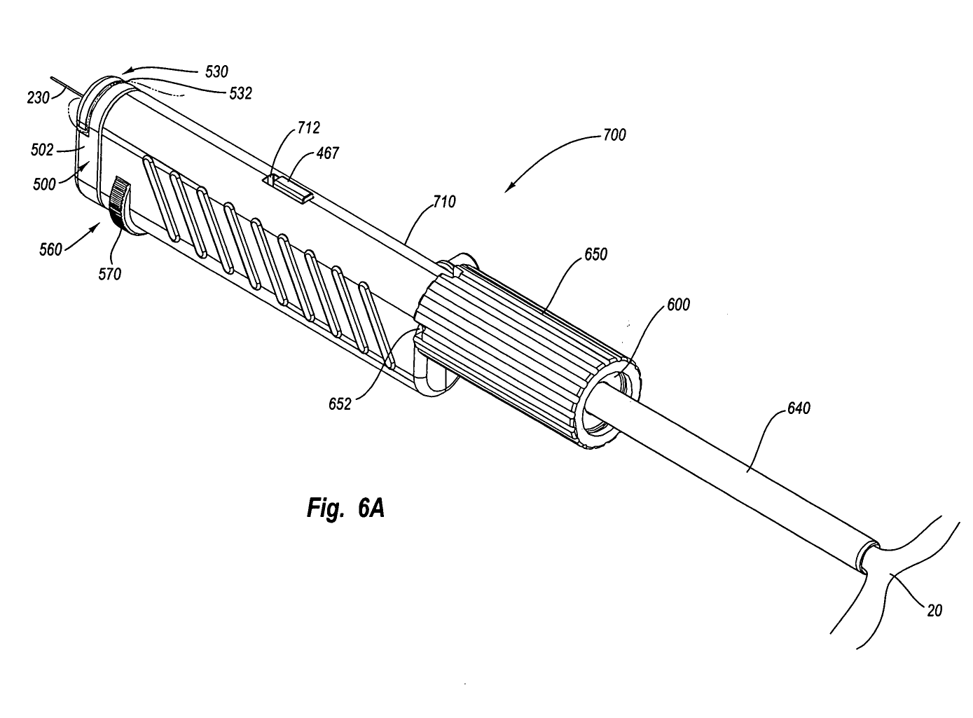

FIGS. 5A-5F depict the steps involved in anastomosing the end of a graft vessel device to a side of a target vessel. Once the end-to-side anastomosis is completed, cutting assembly 745 is detached from compression assembly 760 as shown in FIG. 6B. Then ring device 300 is held while compression assembly 760 is pulled to remove the graft vessel device 55 from compression assembly 760. At this point, graft device 55 appears as shown in FIG. 6C.

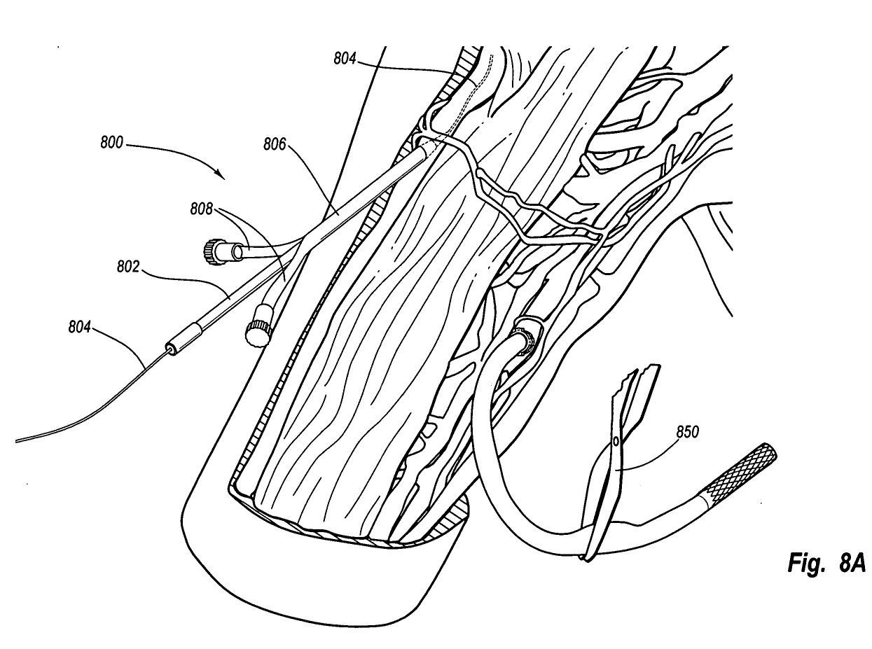

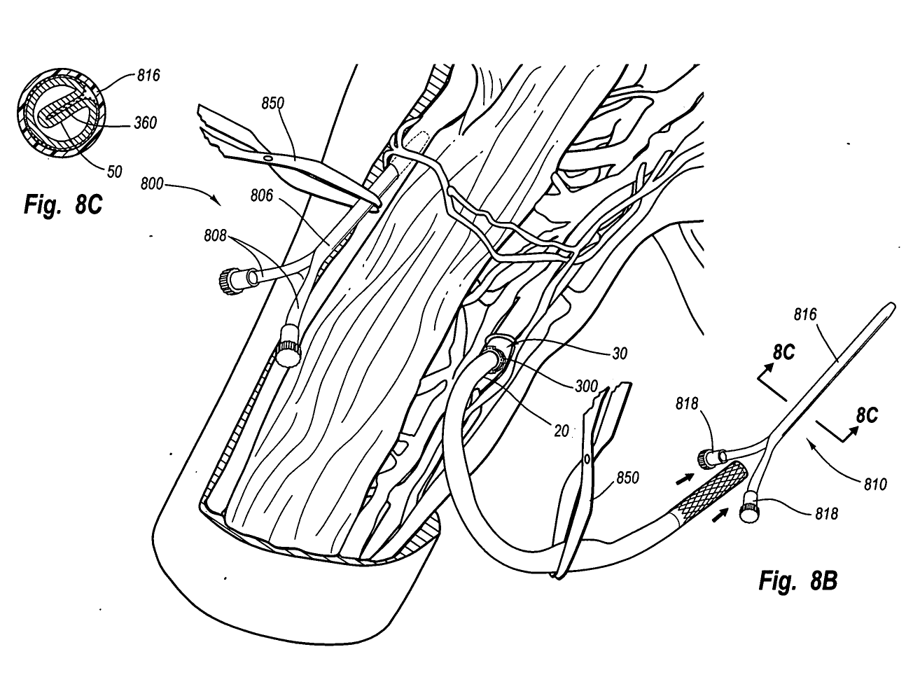

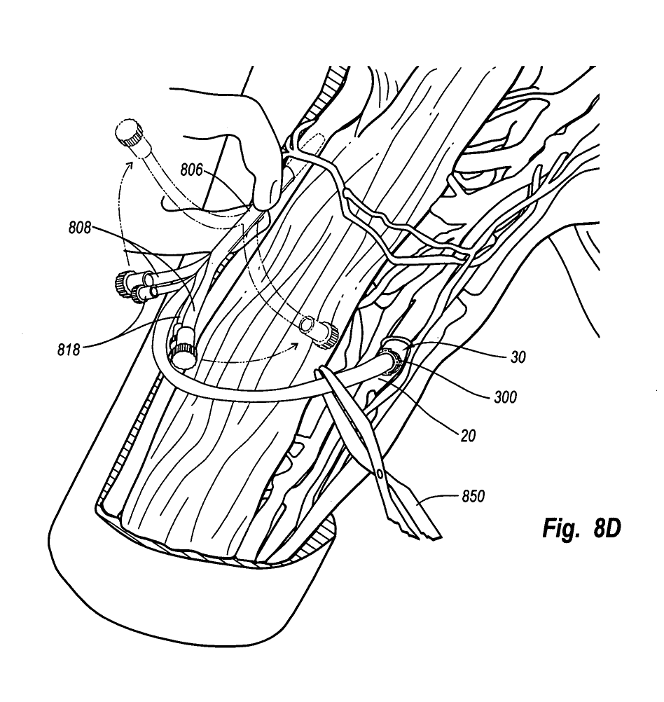

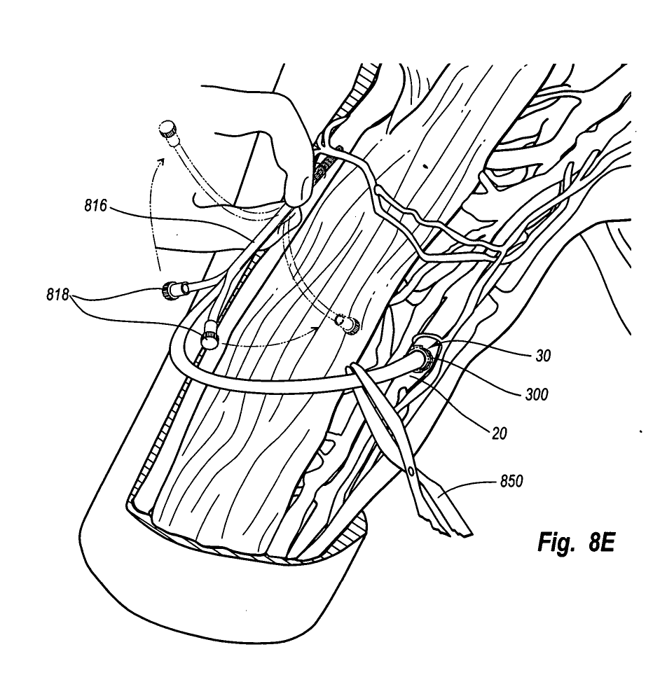

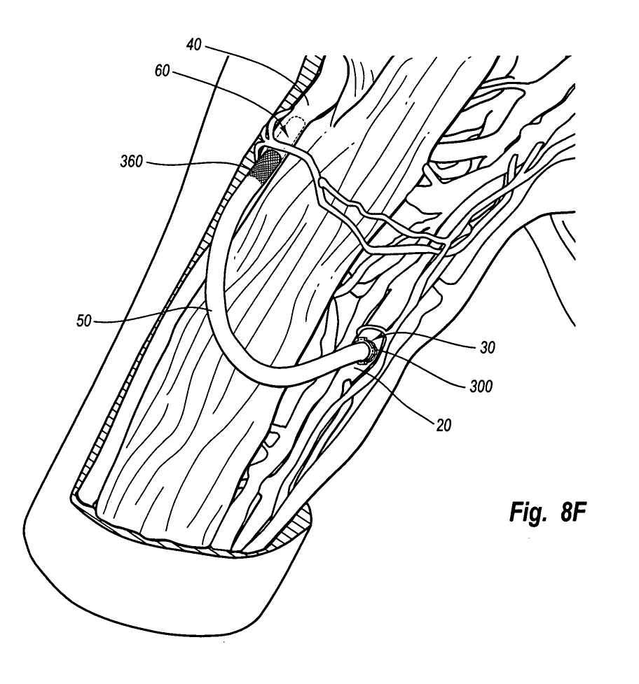

Once the end-to-side anastomosis is completed, then the stented end 60 of graft vessel device 55 is positioned within the lumen of a vessel such as vein 40 shown in FIG. 8F. An exemplary procedure for positioning stented end 60 into the vessel lumen via dilator sheaths is described in reference to FIGS. 8A-8E.

Each of the steps involved in the procedure described above is described below in detail. The devices used to complete these steps are also described below in detail.

To secure a graft vessel in the artery-to-vein configuration depicted in FIG. 1, an anvil apparatus such as anvil apparatus 200 is positioned at an anastomosis site 10 as shown in FIG. 2A. FIGS. 2A-2J depict one procedure for positioning anvil apparatus 200 at anastomosis site 10 using catheter system 100. Once anvil apparatus 200 is positioned at the anastomosis site within lumen 28 and the anastomosis site is externally accessible, then graft 50 is attached to the artery in an end-to-side anastomosis.

As mentioned above, FIGS. 2A-2J depict an exemplary methodology for positioning anvil apparatus 200 at anastomosis site 10. An anastomosis site is first identified by conventional methods such as ultrasound. FIG. 2A depicts a patient undergoing the initial step of a procedure utilized to remotely position the intraluminally directed anvil apparatus 200 at an anastomosis site 10 in a blood vessel 20 (not shown in FIG. 2A) in the chest or arm such as the brachial artery from a catheterization site 40 in a blood vessel in the patient's leg, the femoral artery. The procedure may begin with a micropuncture into the femoral artery followed by introduction of a guide wire. Catheter system 100 is shown in FIG. 1 with an introducer 110 inserted at catheterization site 40 in the femoral artery. Introducer 110 permits a guide wire 120 to be inserted to the anastomosis site. Guide wire 120 preferably utilizes a coil 125 to minimize the potential of the guide wire 120 to cause damage. Guide wire 120 typically follows a fluoroscopic device, an endoscopic device or some other remote viewing instrumentation or imaging technique used to determine the location for the anastomosis site 10 such as the proximity of a blood vessel occlusion or another abnormality that has been detected by a conventional exploration technique. Any conventional guide wire suited for inserting both diagnostic and therapeutic catheters may be utilized.

Hub 115 is shown at the proximal end of guide wire 120 in FIG. 1. The proximal end of a catheter system such as catheter system 100 comprises one or a plurality of access ports or luer fittings such as hub 115. For the purpose of simplicity, the proximal end of the various catheters depicted in FIG. 2A-2I are not shown. However, the manufacture and handling of a catheter system with a plurality of lumens and a plurality of access ports are known to those of ordinary skill in the art.

FIG. 2B depicts a catheter 140 within the lumen of a blood vessel. A guide wire 120 is positioned within catheter 140. Guide wire 120 has a coil 125 at its end of guide wire 120 positioned at the selected anastomosis site 10. FIG. 2C depicts a guide wire loop snare 142 positioned within the lumen of a blood vessel. After a cut down procedure has been performed to expose the target vessel, then a loop snare catheter 144 is introduced as shown in FIG. 2C. FIG. 2D depicts a guide wire 142 snaring a wire 152 which extends from catheter 150. The combination is then pulled back into catheter 140 as shown in FIG. 2E. Wire 152 is then withdrawn from catheter 150 as shown in FIG. 2F to yield the configuration shown in FIG. 2G. The sizes of catheter 140 and catheter 150 may respectively be 12 French and 3 French.

FIG. 2H depicts anvil pull 230 extending through catheter 140 with anvil 210 abutting the end of catheter 140. Anvil pull 230 is then pulled to advance anvil 210 to anastomosis site 10 as shown in FIG. 2I. FIGS. 2H-2J depict an optional wire 240′ extending from anvil 210. Optional wire 240 is referred to herein as a positioning stem or a removal stem 240. Stem enables anvil 210 to be controlled at the opposite end from anvil pull 230.

Other methods for positioning an anvil of an anvil apparatus at an anastomosis site are disclosed in U.S. Pat. No. 6,248,117 titled Anastomosis Apparatus for Use in Intraluminally Directed Vascular Anastomosis and in U.S. Pat. No. 6,652,542 titled Intraluminally Directed Vascular Anastomosis. These patents, which are owned by Integrated Vascular Interventional Technologies, L.C. (IVIT, LC), are both incorporated herein by reference in their entirety

FIG. 2J shows that once anvil apparatus 200 has been positioned at anastomosis site 10 such that anvil pull 230 extends out of blood vessel 20 through initial piercing 15 in the wall of the first vessel then anvil pull 230 can be maneuvered to hold engaging end 212 of anvil 210 against interior 22 of the wall of blood vessel 22. Note that since initial piercing 15 is so much smaller than engaging end 212 of anvil 210, anvil 210 cannot pass through initial piercing 15. This difference in size enables anvil 210 to be pulled against interior 22 in a manner which enables the wall of vessel 20 to be distended. As discussed below, the ability to pull anvil pull 230 such that engaging end 212 of anvil 210 engages interior 22 and distends the wall of vessel 20 contributes significantly to the ability to evert the portions of the vessel wall around an opening or anastomosis fenestra used for attaching another vessel. Anvil 210 also has a cylindrical landing 214 which are its sidewall surfaces that assist in the eversion process as described below in reference to FIGS. 5A-5F.

Anvil apparatus 200 comprises anvil 210 and anvil pull 230 and may also optionally comprise stem 240. Anvil 210 and anvil pull 230 are preferably fixedly attached together. As shown, anvil pull 230 extends through anvil 210 via an anvil aperture 216 (not shown) and optionally terminates at a stopping element (not shown). Since the anvil pull is typically metal and the anvil is typically molded plastic, stopping element 236 may be just the proximal end of anvil pull 230 embedded in anvil 210 such that it is not visible or such that it just slightly extends beyond terminal end 218. Anvil 210 and anvil pull 230 may also be integral. Anvil 210 may also be coated with an elastomeric material. Additionally, anvil 210 may be movably positioned on anvil pull 230 in which case, a stopping element can be used to brace against terminal end 218 of anvil 210.

Other features and configurations for the anvil apparatus are described in U.S. Pat. No. 6,623,494 titled Methods and Systems for Intraluminally Directed Vascular Anastomosis, U.S. patent application Ser. No. 09/736,839 titled Intraluminally Directed Anvil Apparatus and Related Methods and Systems, U.S. patent application Ser. No. 10/003,985 titled Soft Anvil Apparatus for Cutting Anastomosis Fenestra and U.S. Pat. No. 6,626,921 titled Externally Positioned Anvil Apparatus for Cutting Anastomosis. These patents and patent applications, which all owned by Integrated Vascular Interventional Technologies, L.C. (IVIT, LC), are incorporated herein by reference in their entirety.

After the anvil 210 has been positioned such that its engaging end 212 contacts the intima of vessel 20 with anvil pull 230 extending through the wall of vessel 20, then anvil apparatus is ready to be utilized in an anastomosis procedure for joining vessel 20 with another vessel such as graft vessel 50. Graft vessel may be autologous or heterologous, however, it is preferably a synthetic material such as conventional ePTFE tubular grafts.

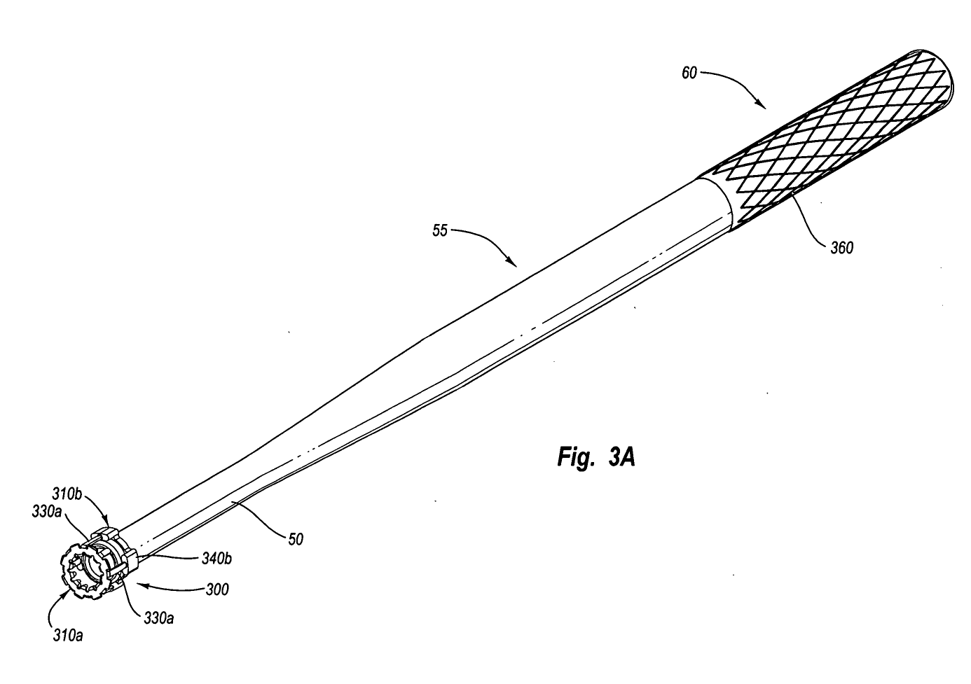

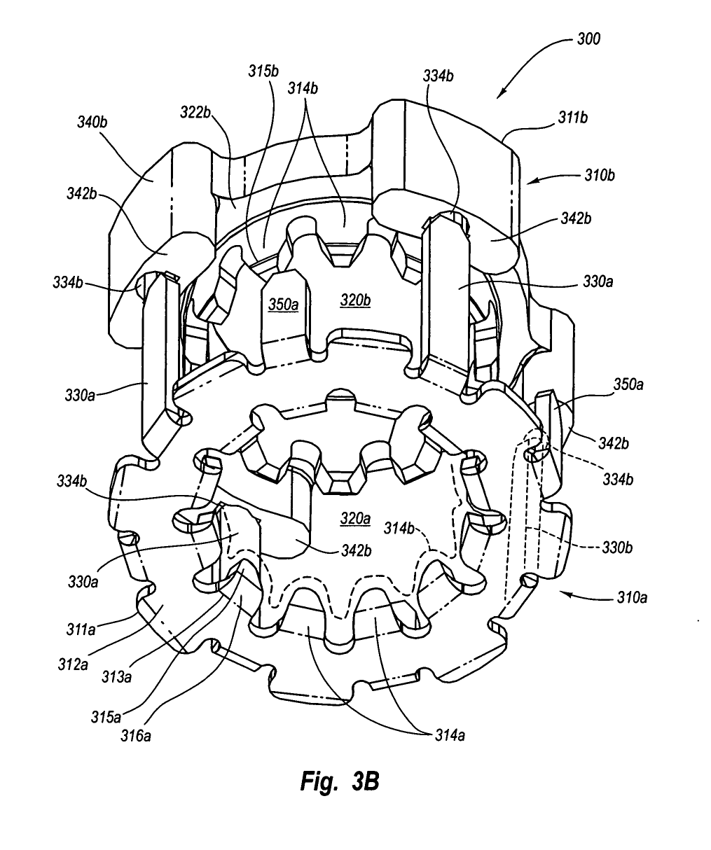

The end-to-side anastomosis is achieved by utilizing anvil apparatus 200, an anastomosis ring device 300 and operator 700. FIGS. 3A-3B depict an embodiment of an anastomosis ring device 300 in detail. FIGS. 5A-5G depict the procedure for attaching ring device 300 through the combined use of anvil apparatus 200 and operator 700. The components of operator 700 and their functions are described in reference to FIGS. 6A-6C and 7A-7F.

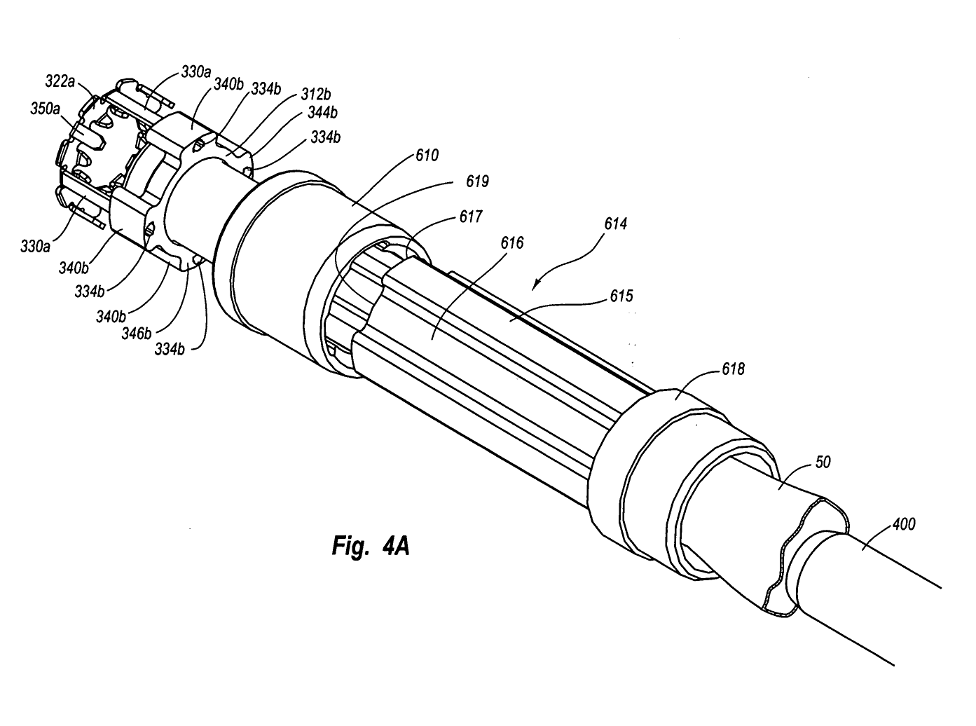

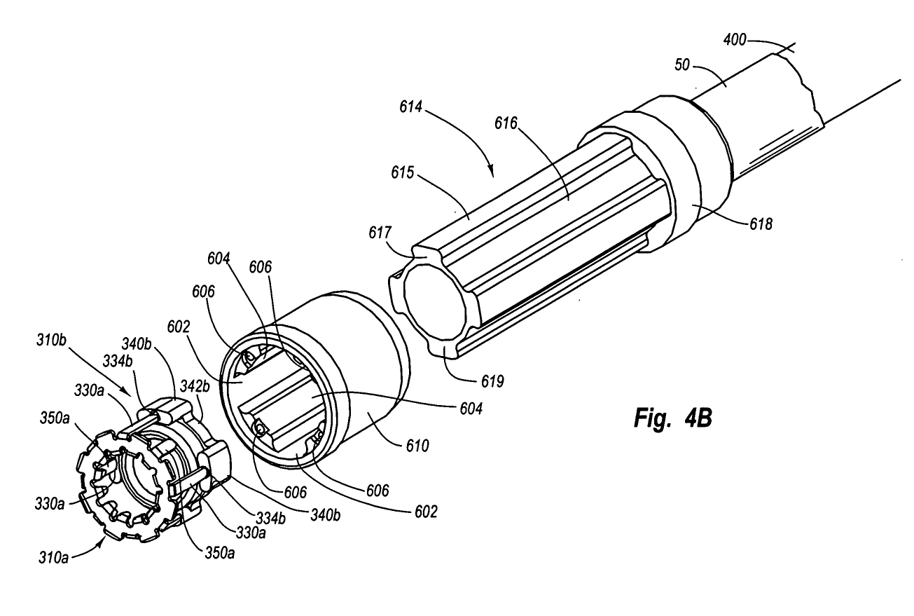

FIG. 3A depicts graft vessel device 55 with greater detail than can be seen in FIG. 1. Graft vessel device 55 comprises a graft vessel 50 with components at both ends for securing the graft vessel to other target vessels. Graft vessel device 55 is shown with an anastomosis ring device 300 at anastomosis end 30 of graft vessel 50 in FIG. 3A. Anastomosis ring device or ring device 300 is shown in greater detail in FIG. 3B. While the features of anastomosis ring device 300 are best seen in FIG. 3B, the functional purposes of these features are best understood with reference to FIGS. 4A-4B and 5A-5G. At the other end of graft vessel device 55, stented end 60, a stent 360 is attached to graft vessel 50. It should be understood that the components at the opposing ends of graft device 55 are exemplary of components which can be utilized to secured the graft vessel to other target vessels. Note also that the term anastomosis component refers to a complete device for achieving anastomosis such as ring device 300 or stent 360 or part of such a device like graft vessel ring 310b.

Anastomosis ring device 300 or ring device 300 comprises a ring 310a and a ring 310b. Ring 310a is referred to as a first ring or a target vessel ring depending on the context. Similarly, ring 310b is referred to as second ring or a graft vessel ring. Target vessel ring 310a and graft vessel ring 310b hold the vessel tissues between them as shown in FIG. 5G once the rings have been approximated. Ring device 300 is shown in FIG. 3A in its unapproximated configuration with graft 50 loaded onto graft vessel ring 310b. Ring device 300 as shown in FIG. 3A is ready to be positioned into operator 700. Note that the position of graft vessel 55 in operator 700 is best seen in FIG. 7D.

Target vessel ring 310b and graft vessel ring 310a are approximated in the embodiment of ring device shown at 300 by pressing graft vessel ring 310a towards target vessel ring 310b as target vessel ring 310b remains stationary. The structure which enables graft vessel ring 310a and target vessel ring 310b to be brought together while target vessel ring 310b remains stationary is described below in reference to FIGS. 4A-4B and FIGS. 5A-5G.

Rings 310a-b are provided in the exemplary embodiment of ring device 300 with a plurality of holding surfaces referred to herein as holding tabs or holding extensions which hold the portion of vessel around its opening. Holding tabs 314a-b respectively protruding from opposing anastomosis sides 322a and 322b of rings 310a-b. More particularly, holding tabs 314a-b extend respectively from ring structures 312a-b of rings 310a-b. Holding tabs 314a-b are intended to hold the everted contours of the vessels being anastomosed. Each one of holding tabs 314a-b has a base 316a-b that integrally extends from the anastomosis side 322a-b of the ring loop 312a-b of the corresponding ring at inner periphery 313a-b and that terminate at rounded tips 315a-b. Distal tips 315a-b are rounded as shown to minimize the potential for penetration. However, in some embodiments, the distal tips may be pointed, for example, when holding a graft vessel. When using tabs with pointed tips care should be used to avoid penetration of the target vessel. Holding tabs 314a-b are typically rather rigid, however, they may also be designed to elastically bend in such a way that the distal tips of such holding tabs slightly swing about their respective bases. Such a bending action may be caused by the displacement through any of openings 320a-b defined by holding tabs 314a-b, more particularly the distal tips 315a-b of holding tabs 314a-b.

The number of holding tabs and their spacing may be varied as needed as long as the portions of the vessels defining the vessel openings can be maintained in an everted orientation. For example, the plurality of holding tabs may include twelve holding tabs as shown in FIG. 3B. However, smaller amounts may also be utilized, for example there may be only six to ten holding tabs.

Holding tabs such as holding tabs 314a-b can have a plurality of shapes. The holding tabs preferably used in embodiments of this invention are wider at the base and so configured as to extend into a distal rounded tip at the end opposite to the base. Although holding tabs 314a-b can be distributed in a variety of arrays, a generally regular distribution on the anastomosis sides of the rings is preferred.

Each of the holding tabs shown in the embodiment depicted in FIG. 3B is attached at its base 316a-b at the inner peripheries 313a-b of rings 312a-b. However, the bases 316a-b may also extend from other locations of the rings. For example, the bases 316a-b may extend from ring loops 312a-b between the outer peripheries 311a-b and the inner peripheries 313a-b or perimeter on the anastomosis sides 322a-b of each ring. The holding tabs disclosed herein are examples of holding surfaces.

All of the holding tabs disclosed herein are also examples of holding means for holding the first vessel at the first vessel opening. Also all of the rings disclosed herein are examples of ring means for providing support for vessel at the opening of the vessel. Additionally, he ring devices disclosed herein are all examples of means for joining a portion of the first vessel that defines the first vessel opening to a portion of a second vessel that defines a second vessel opening.

After the vessel tissue is everted onto the rings, the anastomosis is formed by bringing the everted interior of the graft vessel into contact with the everted, interior portion of the target vessel. Because the tissue is held together in this everted configuration, with the interior of one vessel compressed against the interior of the other vessel, there is no foreign material exposed to the interior of the vessel.

In the embodiment of the ring device identified at 300, the holding tabs of each ring are positioned so that they may interdigitate with the holding tabs of the other ring. Stated otherwise, the rings may be oriented so that when the rings are brought together, each holding tab of one ring is opposite the space between two neighboring holding tabs in the opposing ring. The everted tissue held together between the interdigitated holding surfaces creates a secure anastomosis. The leading edges of the holding tabs act as the rim of each ring and stretch the material. One advantage of this configuration is that the interface between these everted, interdigitated vessels is not flat as it would be if the interface was between leading edges which are essentially two round rims. A flat interface is more susceptible to inaccuracies in alignment which prevent a fluid tight configuration. The varied, wavy interface at the anastomosis of the two vessel openings 24 and 54 shown in FIG. 5G provides a better ability to create a seal. The stretching of the tissue as it conforms to the shape of the tabs and the spaces between the tabs assists in creating a seal. Conformance to the interdigitated configuration also provides some overlap of tissue. The varied, wavy interface also results in an uneven distribution of force against the portions of the vessel which define their respective openings. An advantage of an uneven distribution of force is a decrease in the likelihood of necrosis occurring symmetrically around the perimeter of the target vessel opening which would likely cause the anastomosis to fail.

Other examples of interdigitated or mated configurations of anastomosis rings or plates are provided in U.S. patent application Ser. No. 10/035,084 titled Paired Expandable Anastomosis Devices which was filed on Dec. 27, 2001 on behalf of Duane D. Blatter, Michael C. Barrus, and Troy J. Orr; U.S. patent application Ser. No. 09/737,200 titled Ring Anastomosis Apparatus and Related Systems which was filed on Dec. 14, 2000 on behalf of Duane D. Blatter, Kenneth C. Goodrich, Michael C. Barrus, and Bruce M. Burnett; U.S. patent application Ser. No. 09/736,937 titled Locking Anastomosis ring device which was filed on Dec. 14, 2000 on behalf of Duane D. Blatter, Kenneth C. Goodrich, Michael C. Barrus, and Bruce M. Burnett; and U.S. Pat. No. 6,569,173 titled Ring Anastomosis Apparatus which was filed on Dec. 14, 1999 on behalf of Duane D. Blatter, Kenneth C. Goodrich, Mike Barrus, and Bruce M. Burnett. Additional ring configurations are disclosed in U.S. patent application Ser. No. 10/624,315 titled Apparatus and Methods for Facilitating Repeated Vascular Access which was filed on Jul. 21, 2003 on behalf of Duane D. Blatter and U.S. patent application Ser. No. 10/351,172, which was filed on Jan. 23, 2003 on behalf of Duane D. Blatter, Troy J. Orr and Michael C. Barrus. These patent and patent applications, which are owned by Integrated Vascular Interventional Technologies, L.C. (IVIT, LC), are incorporated herein by reference in their entirety.

As indicated above, interdigitated holding surfaces are achieved when a holding tab of one ring is opposite the space between two neighboring holding tabs in the opposing ring. As shown by the phantom lines in FIG. 3B, holding tabs 314b are offset from holding tabs 314a such that as the rings are brought towards each other each holding tab 314b is positioned opposite from the spaces between holding tabs 314a in a mated configuration. The rings may be brought together such that the tips 315a-b are not yet in the same plane (the tips of the holding tabs have not entered the opposing spaces between the holding tabs of the other ring), in the same plane, or extend beyond the plane defined by the tips (the tips of the holding tabs have entered the opposing spaces between the holding tabs of the other ring). The spacing required for successful anastomosis varies depending on the types of vessel being anastomosed.

The rings are pushed together until they are sufficiently close that the everted tissue is held in place and the anastomosis is secure. Failure to bring the rings sufficiently close together such that the tips 315a-b are significantly close together risks the potential loss of the tissue that has been captured and everted onto holding tabs 314a-b. It is advantageous when anastomosing a graft vessel to an artery in an end-to-side anastomosis to compress the rings such that holding tabs 314b enter the space between adjacent holding tabs 314a. Such further compression is advantageous to the extent that it is achieved without penetrating blood vessel 20 in a manner that risks failure of the anastomosis.

An example of a suitable compression is provided by an anastomosis ring device having holding tabs with lengths of 0.045 inches (0.1143 cm) that has a distance between the anastomosis sides 322a-b of rings 312a-b of 0.090 inches (0.2286 cm). Compression down to only 0.10 inches (0.254 cm) for such a anastomosis ring device may not be sufficient to hold the anastomosed tissues. The rings may be further compressed such that the distance between the anastomosis sides 322a-b is 0.080 inches (0.2032 cm) or 0.070 inches (0.1778 cm) to bring vessel 20 and vessel 50 even closer together. However, as noted above, it is preferable to avoid pushing through the vessels. The rings are accordingly designed to permit compression down to the ideal spacing between the anastomosis sides while providing holding tabs that are long enough to capture the tissue in an everted configuration.

The ring device has a locking configuration that maintains two rings in a desired spatial relationship so that the anastomosis is secure. The locking configuration maintains the holding tabs or other holding surfaces close enough together to maintain a secure, substantially leak proof anastomosis. The locking configuration also maintains the holding surfaces sufficiently separated from each other to avoid necrosis of the native tissue involved in the anastomosis. In one embodiment, the locking configuration is provided by guideposts extending from one ring that are adapted to cooperate with guides or guide receptacles in the other ring. The guideposts frictionally engage the guide receptacles so that the holding surfaces of the rings are locked together. Ring device 300 has such a configuration. The combination of guideposts and guide receptacles are examples of locking means for locking the first ring and second ring together such that the first vessel and the second vessel remain anastomosed together.

Target vessel ring 310a has a plurality of guideposts 330a extending from its ring loop 312a which frictionally extend in guide receptacles 334b of graft vessel ring 310b. Note that each guide post housing 340b defines a guide receptacle 334b and sheaths the corresponding guidepost 330a. Guideposts 330a permit the relative approach of these two rings as graft vessel ring 310b is driven forward on guideposts 330a towards ring 310a. More particularly, guideposts 330a enable rings 310a-b to be brought together in a manner such that graft vessel ring 310b is moved in a fixed parallel orientation relative to target vessel ring 310a. Additionally, guideposts 330a are positioned relative to holding tabs 314a-b and have a length that permits graft vessel 50 to be loaded onto holding tabs 314b and then be brought into contact with blood vessel 20. Stated otherwise, the configuration of guideposts 330a enables first vessel opening 24 and second vessel opening 54 to be initially spaced apart and opposite from each other and then to be advanced toward each other as graft vessel ring 310b is moved with graft vessel 50 held on the holding tabs 314b while blood vessel 20 is held by holding tabs 314a of target vessel ring 310a.

Several factors enable guideposts 330a to be securely retained in guide receptacles 334b of graft vessel ring 310b. Ring 310a may be formed from a material which is harder or has a higher compressive modulus (as measured by ASTM D695) than the material used to form ring 310b or at least parts of ring 310b. For example, ring 310a may be formed from non-magnetic stainless steel such as 316L stainless steel while ring 310b is formed from a hard biocompatible plastic material. Such a configuration enables steel guideposts 330a to be driven into the softer plastic which defines guide receptacles 334b and deform the plastic to the extent that guideposts 330a differ in diameter and/or cross-sectional shape relative to guide receptacles 334b. Examples of suitable hard biocompatible plastic material include nylon, polyetheretherketone (PEEK), and ultra high molecular weight (UHMW) polyethylene. In addition to selecting an appropriate compressive modulus for each of the two materials used to form guideposts and the guide receptacles, the ability of guideposts 330a to be securely retained can be increased by using geometries and cross-sectional shapes which are different form each other. For example, a rectangular guideposts can be pushed into a round guide receptacle. As explained below, the amount of force required to position guideposts within guide receptacles based on the materials, geometries, cross-sectional shapes, etc., is carefully selected.

FIGS. 4A-4B are partially exploded perspective views which depicts guideposts 330a extending slightly into guide receptacles 334b before the rings have been compressed together. As shown, ring device 300 has a plurality of guides. While ring device 300 is shown with four guides 330a, other embodiments may have other configurations such that the plurality of guides includes, for example, three to six guides. It is even possible to have only one guide. Although guides 330 can be distributed in a variety of arrays, a generally regular distribution provides for easiest approximation of the rings in a parallel configuration.

In addition to various arrays and variable numbers, the guideposts may have a variety of lengths. The guideposts may also extend from one or both of the rings at any appropriate location. Guideposts 330a are situated such that the portion 27 defining the blood vessel opening 24 and the portion 57 defining the graft vessel opening 54 are joined without being penetrated as the first vessel and the second vessel are anastomosed together. The guideposts and guide receptacles disclosed herein are exemplary embodiments of means for locking one ring with respect to the other ring.

Target vessel ring 310a is retained in a fixed position relative to operator 700 by ring retainer 610. Ring 310a has retention prongs 350a which are positioned in retention receptacles 606 of ring retainer 610. Ring retainer 610 is fixedly secured in the distal end of tubular housing 640 of operator 700 as best seen in FIG. 5A and FIG. 7A. Ringer retainer 610 has tracks 604 separated by grooves 602.

Approximator 614 has tracks 615 separated by grooves 616. Tracks 615 of approximator 614 are positioned in grooves 602 of ring retainer 610 and tracks 604 of ring retainer are positioned in grooves 616 of approximator 614. This mated configuration of the tracks and grooves of ring retainer 610 and approximator 614 permit the approximator 614 to be advanced through ring retainer 610 and to be driven against graft vessel ring 310b.

Approximator 614 has the same cross-sectional shape or profile as graft vessel ring 310b. This enables the distal end 619 of approximator 614 to be driven against the proximal side 346b of graft vessel ring 310b. More particularly, bearing face 617 at the distal end of each track 615 of approximator 614 and the rim around the opening at the distal end 619 of approximator 614 as shown in FIG. 4B are driven against the bearing surface 344b of guide receptacle housing 340b and ring loop 312b on the proximal side 346b of graft vessel ring 310b as shown in a FIG. 4A.

Approximator 614 has a shoulder 618 which rides against the interior surface of tubular housing 640. Shoulder 618 provides an abutting surface for movement of approximator 614 within tubular housing 640. Shoulder 618 can also act as a stop for movement of approximator.

FIGS. 4A-4B also show a graft vessel 50 as it extends within approximator 614. FIGS. 4A-4B also show cutter 400 extending within graft vessel 50. Cutter 400 includes a cutting tube that terminates at a cutting knife with a cutting edge 414. Cutting edge 414 is identified in FIG. 5A and is shown in other figures. The cutter and other cutting devices disclosed in the references incorporated by reference are examples of cutting means for forming an opening in the wall of the first vessel at the anastomosis site.

FIGS. 5A-5G depict the sequential movement of graft vessel ring 310b toward target vessel ring 310a to bring the portion of graft vessel 50 that defines the second vessel opening 54 into contact with the portion of blood vessel 20 that defines the first vessel opening 24 such that the blood vessel and the graft vessel are anastomosed together. FIGS. 5A-5G depict the use of anvil apparatus 200, anastomosis ring device 300, cutter 400, and operator 700. To optimally present this sequence, FIGS. 5A-5G are cross-sectional views.

The components of operator 700 shown in FIGS. 5A-5F include tubular housing 640, slide tube 620, approximator 614, and ring retainer 610. As mentioned above, ring retainer 610 is fixedly positioned in the opening of tubular housing 640. Approximator 614 is advanced within tubular housing 640 when it is urged forward by slide tube 620. The components of operator 700 which advance slide tube against approximator 614 are explained below in relation to FIG. 7B. Approximator 614 and slide tube 620 may be a single integral component. Similarly, tubular housing 640 and ring retainer 610 may also be a single integral component. However, these components are presented as separate components as it is easier to form the more complex parts such as ring retainer 610 and approximator 614 from plastic and the simple tubular shapes of tubular housing 640 and slide tube 620 from a metal such as stainless steel.

FIG. 5A depicts anvil 210 in contact with the intima or interior of the vessel in lumen 28 of vessel 20. More particularly, FIG. 5A depicts anvil pull 230 positioned at an anastomosis site 10 with anvil pull 230 extending through the initial piercing 15 in the wall of the target vessel 20. Operator 700 is shown being positioned at anastomosis site 10. Anvil pull 230 extends concentrically though openings 320a-b of target vessel ring 310a and graft vessel ring 310b. Anvil pull 230 also extends through cutter 400. Cutter 400 extends within graft vessel 50 through slide tube 620 and approximator 614. Cutting edge 414 of cutter 400 extends beyond graft vessel ring 310b toward target vessel ring 310a.

FIG. 5B depicts anvil 210 being pulled against the vessel wall such that vessel 20 is sufficiently distended to permit the vessel 20 at anastomosis site 10 to be pulled into anastomosis ring device 300 through first ring opening 320a. More particularly, anvil 210 is pulled by anvil pull 230 such that all of spherical engaging end 212 is pulled into the anastomosis ring device 300 and part of cylindrical landing 214. Anvil 210 has advanced sufficiently into anastomosis ring device 300 to enable cutter 400 to contact the portion of the blood vessel 20 distended by anvil 210.

FIG. 5C depicts the formation of a first vessel opening or a target vessel opening 24 in the wall of the first vessel. Target vessel opening 24 is formed by pulling anvil pull 230 against cutter 400 with sufficient force to enable anvil 210 to advance blood vessel 20 against cutting edge 414. After the cut has been made then a cut portion 25 of the wall of blood vessel 20 remains on spherical engaging end 212 of anvil 210 while the portion 26 of the blood vessel around first vessel opening 24 rest on anvil landing 214. Note that after the vessel is cut, the vessel retracts as it is no longer being stretched.

As described below in relation to FIG. 7B, cutter 400 is spring biased. The load of the spring can range from 0 up to about 12 lbs but it is typically about 4 lbs. Anvil pull 230 applies additional force ranging from about 4 lbs to about 12 lbs but it is typically about 8 lbs. Cutting typically occurs within the range of 7-12 lbs. Spring biasing cutter 400 reduces the amount of force needed to urge anvil 210 against cutter 400.

In addition to reducing the amount of force used to pull anvil pull 230, spring biasing cutter 400 impacts the ability to cut target vessel 20 at a desired position within anastomosis ring device 300. It is desirable to cut vessel 20 at a position within ring device 300 which permits portion 26 of vessel ring 20 to be everted over holding tabs 314a. By drawing anvil 210 into ring device 300 and stretching vessel 20, the size of the target vessel opening is minimized and portion 26 of the vessel which rests on landing 214 of anvil 210 has enough length to be everted over holding tabs 314a.

Note that vessel 20 is held in between holding tabs 314a and landing 214 of anvil 210 after the cut is made to maintain the length of the tissue resting on landing 214 and to prevent the tissue from escaping from between these two structures. By holding the vessel tissue between holding tabs 314a and landing 214 of anvil 210 to mechanically capture portion 26, blood flow out of the target vessel opening is substantially prevented or at least minimized. So the flow of the blood during this phase of the procedure is controlled by controlling the tissue. For this reason, the length of landing 214, the offset between rings 310a-b (controlled by the length of guide posts 330a), the position of cutter 400 within ring device 300 and the spring biasing of cutter 400 are all optimized to enable the portion of the target vessel resting on landing 214 to have sufficient length to be everted on holding tabs 314a. Another factor to be considered when optimizing the length for eversion is the amount of vessel retraction after the vessel has been cut and is no longer stretched. It should also be noted that excessive lengths should be avoided as portions which are too long may be difficult to flip into an everted configuration.

The eversion does not need to be achieved around the entire perimeter of the target vessel ring as long as hemostasis is achieved. However, the effectiveness of the anastomosis increases as more of the tissue is everted around the perimeter of the opening of the target vessel in conformance with the perimeter opening of the target vessel ring.

FIG. 5D depicts ring device 300 after compression of graft vessel ring 310b toward target vessel ring 310a. FIG. 5D shows portion 26 everted over holding tabs 314a. Eversion of portion 26 is achieved by advancing graft vessel ring 310b toward target vessel ring 310a. By advancing graft vessel ring 310, the portion of graft vessel 50 everted onto holding tabs 310b is driven on landing 214 against portion 26 of target vessel 20. As the portion of graft vessel 50 everted onto holding tabs 310b is driven on landing 214, it causes portion 26 to buckle and then flip over holding tabs 314a. So the eversion is achieved at the same time that graft 50 is brought together with target vessel 20.

As indicated above, the compression of ring device 300 as shown in FIG. 5D is achieved by moving graft vessel ring 310b toward ring 310a via force from slide tube 620 against approximator 614. As the distal end 619 of approximator 614 is driven against the proximal side 346b of graft vessel ring 310b, guide receptacles 334b are pushed on guide posts 330a. FIG. 5E shows further compression of ring device 300. Note that guide posts 330a now extend the entire length of guide receptacles 334b while FIG. 5D shows guideposts 330a before full compression. Guide receptacles 334b are shown with open ends, however, the guide receptacles could also have a closed end so that it is not possible for the guideposts to penetrate out of the guide receptacles. When guide posts 330a are positioned fully into guide receptacles 334b, the rings 310a-b are securely locked together with a desired offset.

FIG. 5F depicts the release of target vessel ring 310b from ring retainer 610. As discussed above with reference to FIG. 4B, vessel ring 310a is retained in a fixed position relative to operator 700 by ring retainer 610. More specifically, retention prongs 350a of target vessel ring 310a are releasably retained by retention receptacles 606 of ring retainer 610. Note that retention prongs 350a of target vessel ring 310a shown in FIG. 4B and FIG. 5A-5D are not visible in the view depicted in FIG. 5F due to the particular cross-section shown. During the process of compressing ring device 300, force is applied to move graft vessel ring 310b toward target vessel ring 310a by pushing the distal end 619 of approximator 614 against the proximal side 346b of graft vessel ring 310b. As guide receptacles 334b are pushed on guide posts 330a in this process, force is also applied to displace retention prongs 350a of target vessel ring 310a out of retention receptacles 606 of ring retainer 610. However, the force is not adequate to move retention prongs 350a out of retention receptacles 606. After guide receptacles 334b have been fully pushed on guide posts 330a, force applied to graft vessel ring 310b causes further compression of the tissue of the two vessels. This force is then transferred to retention prongs 350a until sufficient force is applied to displace retention prongs 350a from retention receptacles 606 of ring retainer 610. When this level of force is applied, ring device 300 is then pushed out of applicator 700 by approximator 614. As described below, the amount of force required to eject ring device 300 is selected to coincide with the amount of force desired for approximating the vessel openings together as they are held by the respective holding tabs.

Note that retention prongs 350a are shorter than guide posts 330a. Retention prongs 350 are extend along recesses 342b between guide receptacle housing 340b. This configuration minimizes the likelihood that guide posts 330a will penetrate the target vessel or other surrounding tissues.

When configuring ring device 300 and ring retainer 610 such that greater force is required to push retention prongs 350a out of retention receptacles 606 than is required to push guide receptacles 334 on to guide posts 330a, several factors are balanced. Enough force is applied to ensure interdigitation and hemostasis but not so much that excessive penetration occurs. Excessive penetration or excessive compression of the vessel can limit blood flow to the region and result in necrosis. Ring device 300 and ring retainer 610 may be designed to enable retention receptacles 606 of ring retainer 610 to continue holding retention prongs 350a until at least about 12 lbs is applied or up to about 20 lbs for even greater compression of rings 310a-b. Ring device 300 may be designed so that the amount of force required to drive guide receptacles 334b on to guide posts 330a ranges from about 6 lbs to about 8 lbs. Systems designed within these ranges enable the tissue of the vessels to be held together with a force ranging from about 2 to about 12 lbs. Sutures typically can withstand about 2 lbs of pressure so anastomosis ring device 300 provides a much stronger anastomosis. An example of a suitable system is a design which requires about 12 lbs force to displace retention prongs 350a from retention receptacles 606 of ring retainer 610 and about 8 lbs to push guide receptacles 334b on to guide posts 330a ranges. Such a system provides about 4 lbs of compression to the tissue held between holding tabs 314a-b.

The retention of retention prongs 350a by retention receptacles 606 is achieved in the same manner as the retention of guide posts 330a by guide receptacles 334b. For example, ring retainer 610 may be formed from a material which is softer or has a smaller compressive modulus (as measured by ASTM D695) than the material of retention prongs 350a. More particularly, ring retainer 610 may be plastic while retention prongs are formed from a metal such as stainless steel. Retention prongs 350a may also have a different cross-sectional shape or diameter as compared with retention receptacles to ensure that a particular amount of forces is required to push retention prongs 350a out of retention receptacles 606.

By configuring ring device 300 and ring retainer 610 such that greater force is required to push retention prongs 350a out of retention receptacles 606 than is required to push guide receptacles 334 on to guide posts 330a, several advantages are achieved. As noted above, over compression of the tissue of the vessels is avoided by designing the systems so that ring device 300 is released from ring retainer 610 of applicator 700 at a particular level of force. Another advantage is the ability to push ring device 300 out of applicator at essentially the same point when the desired level of compression is achieved. It also allows the compression to be achieved within applicator 700.

An additional advantage is that the movements are all achieved concentrically within applicator 700. As detailed below, the components of operator are advanced concentrically on a common axis while sheathed in tubular housing and cause the parallel compression of rings 310a-b in a perpendicular relationship to the their common axis. As a result, a blood vessel can be accessed with a minimal cut-down to create the anastomosis. The area of the vessel which requires exposure may be as small as about 20 mm.

As shown in FIG. 5G, after anastomosis ring device 300 has been compressed to join portion 27 of blood vessel 20 that defines first vessel opening 24 to portion 57 of second vessel 50 that defines graft vessel opening 54 then first vessel 20 and second vessel 50 are anastomosed together and are in fluid communication. Anvil apparatus 200 and operator 700 have been removed upon the completion of the procedure through lumen 58 of graft vessel 50. More particularly, once the anastomosis is completed then anvil pull 230 is pulled so that it draws anvil 210 through openings 320a and 320b of anastomosis ring device 300 such that anvil apparatus 200 is removed along with cutter 400 and the other components of operator 700 shown in FIGS. 5A-5F through lumen 58.

Reference is made above to portions 27 and 57 which are held between the respective holding tabs 314a-b and define their respective vessel openings 24 and 54. Note that portions 27 and 57 are also everted over holding tabs and are continuous with portions 26 and 56 which are now the terminal ends of the vessels.

There are also other significant advantages to combining vessels in accordance with the methodology described above especially in a manner such that there is at least partial eversion, contact between the everted surfaces and no penetration of the portions of the vessels defining the vessel openings. Of course, the anastomosis is fluid tight to normal systolic pressure and remains intact under stress. Since the everted portions 27 and 57 respectively cover the holding tabs 314a-b, no intraluminal foreign material is exposed and intraluminal exposure of subintimal connective tissue is minimized. As a result, the thrombogenicity of the anastomosis is no greater than that of hand sutured anastomosis. Additionally, the configuration also results in an anastomosis that is morphologically satisfactory, including eversion of the receiving blood vessel intima with apposition to graft vessel. Further, everted portions 27 and 57 are in intima-intima contact and no cut portion is significantly exposed to the blood flow that is to circulate through the anastomosis.

In addition to the results achieved, there are also significant procedural advantages. The method does not require temporary occlusion of blood flow to the target blood vessel. The anastomosis can be reliably created. Additionally, the anastomosis is rapidly achieved and eliminates the need for high skilled suturing. For example, once the anvil pull extends through the wall of the vessel, the anastomosis procedure can be accomplished in as little as 30 seconds when rings are used to join the vessels.

FIG. 5G shows one of the numerous inventive features disclosed herein related to joining a portion of a first vessel that defines a first vessel opening to a portion of a second vessel that defines a second vessel opening such that the first vessel and the second vessel are anastomosed together and are in fluid communication. Target vessel opening 24 and graft vessel opening 54 have an interface which is wavy or has a sinusoidal shape. This wavy sinusoidal shape is caused by the interdigitation of holding tabs 314a-b which then causes the interdigitation of the portions of the vessels which define their respective openings. The advantages of such an interdigitated configuration are discussed above.

FIG. 6A provides a perspective view of external anastomosis operator 700 with anvil pull 230 positioned in operator and pulling the anvil into the compression ring device held in the anastomosis end of operator 700. The features and configurations of the elements of operator 700 are described in greater detail below in reference to FIG. 7B and FIG. 7D. However, FIG. 6A identifies some of the elements of operator 700 including body 710 and tubular housing 640.

FIG. 6A shows the distension of target vessel 20 as anvil 210 is pulled into operator 700. Anvil pull 230 extends through tubular housing 640, translator 600, actuator knob 650 and body 710. Anvil pull 230 is shown in FIG. 6A extending beyond the end of operator 700 out of anvil pull engager 500. Note that as described in more detail with regard to the FIG. 6C, anvil pull engager 500 includes anvil pull holder 530 and anvil pull advancer 560 which are linked via coupler 502.

FIG. 6B shows the separated configuration of operator 700 into its cutting assembly 745 and its compression assembly 760. After vessels 20 and 50 have been anastomosed together, then the cutting assembly 745 and the compression assembly 760 are separated to permit the removal of graft vessel device. Stented end 60 of graft vessel device 55 is shown extending out of the proximal end of tubular housing 640. Cutting assembly 745 and compression assembly 760 are separated by removing the separator pin 642 which couples tubular housing 640 to body 710. Note that separator pin 642 has been reattached in FIG. 6B. Note also that graft vessel device 55 is not shown being pinched to prevent the flow of blood out of its stented end. It may be initially pinched by finger pressure and then a clamp or other appropriate device may be positioned to prevent the flow of blood. Heparanized saline solution can also be delivered into graft device 55 to keep the graft device flushed.

Once cutting assembly 745 is detached from compression assembly 760 as shown in FIG. 6B, compression assembly 760 is pulled off of graft vessel device 55. Care should be taken to avoid creating tension at the anastomosis site as compression assembly 760 is removed. After compression assembly 760 has been removed then graft vessel device 55 appears as shown in FIG. 6C with its anastomosis end 30 attached in an end-to-side anastomosis with target vessel 20 and its stent end 60 still unattached.

Other components are also shown in FIGS. 6A-6B, however, they are best understood with reference to FIGS. 7A-7F. Actuator knob 570 is shown which enables anvil pull engager 500 to hold anvil pull 230 via anvil pull holder 530 (specifically slot 532) and to advance anvil pull 230 relative to cutter 400 via anvil pull advancer 560. Actuator knob 650 is also shown which enables rings 310a-b to be driven together within tubular housing 640. Actuator knob is shown around translator 600. Rotation of actuator knob 650 linearly advances translator 600 which then drives other components forward which are positioned within tubular housing to compress rings 310a-b together as shown in FIGS. 5A-5F.

FIG. 7A provides a view into tubular housing 640 of applicator 700. Fixedly positioned within tubular housing is ring retainer 610. While not visible from this view, graft vessel ring 310b is glidably mounted in a mated configuration within ring retainer 610. Loaded on graft vessel ring 310b is target vessel ring 310a. Cutter 400 is barely visible within tubular housing 640 in the view provided by FIG. 7A. Cutter 400 is concentrically positioned within tubular housing 640.

FIG. 7A also shows safety indicator 467 and safety extension 468 which are linked together within body 710. A description is provided below in reference to FIG. 7B and FIGS. 7D-7F of the configuration of these components and their ability to prevent compression of the rings before the target vessel is cut.

FIG. 7B is an exploded perspective view of operator 700 with body 710 opened. In this view, the path of anvil pull 230 can be viewed as it extends through cutter 400, safety interlock 466, spring mount 456, spring 460, jam screw 464, guide 536 and out of coupler 502 of anvil pull engager 500 via an anvil pull aperture. The anvil pull aperture and the slot 542 used to secure anvil pull 230 are shown in FIG. 7C.

Anvil pull 230 passes through several other components in addition to those mentioned above. Once anvil pull 230 extends out of operator 700 via holder guide 536 then it is wrapped around slot 532. Note that holder guide 536 may have a cone shaped opening for easy insertion of anvil pull 230.

Slot 532 is located in the upper portion of coupler 502 which is referred to as an anvil pull holder portion. The lower portion of anvil pull engager 500 is referred to as an anvil pull advancer portion. Coupler 502 functions to couple the components which hold anvil pull 230 and then advance anvil pull 230 against cutter 400. Note that the upper portion of coupler 502 referred to above as an anvil pull holder portion is part of anvil pull holder 530 and the lower portion of coupler 502 referred to above as an anvil pull advancer portion is part of an anvil pull advancer 560.

Advancer or drive screw 562 fixedly extends from coupler 502. More specifically, drive screw 562 extends from an advancer guide 568 which is a cylindrical protrusion from the anvil pull advancer portion of coupler 502. Threadably mounted to advancer screw 562 is advancer knob 570. Advancer knob 570 extends out of body 710 via knob slot 550. Advancer knob has a contact post 578 which is a cylindrical extension positioned to rotate in bearing recess 572 as advancer knob 570 is turned. Alignment of coupler 502 is maintained by holder guide 536 within guide receptacle 463 and advancer guide 568 within guide receptacle 552. Advancer screw 562 extends within screw recess 572.

Rotation of advancer knob 570 causes advancer screw 562 to move which in turn causes coupler 502 to move. Since anvil pull 230 is attached to the anvil advancer portion of coupler 502 at slot 532, movement of coupler 502 causes anvil pull 230 to move.

The end of cutter 400 opposite from its cutting edge 414 is positioned within a cutter cup 458 of a safety interlock 466. Safety interlock 466 has a spring mount 456 extending opposite from cutter mount 458. Spring 460 is positioned on spring mount 456 and is positioned within spring recess 462. Spring 460 abuts a jam screw 464 held in a knife tensioner 452 in a threaded engagement. Knife tensioner 452 is held in knife tensioner recess 467 which is in between spring recess 462 and jam screw recess 465.

Obviously spring 460 has more than one variable which impacts its tension. Two of these variables include the inherent tension of spring 460 and the tension of spring 460 as caused by the position of threaded jam screw 464 in knife tensioner 452. Spring 460, knife tensioner 452 and jam screw 452 are an example of a spring biasing device. The spring biasing device is an example of spring biasing means for providing tension against the cutter as the cutter engages the anvil of the intraluminally directed anvil apparatus.

The tension of spring 460 against cutter 400 is overcome as movement of advancer knob 570 causes anvil pull 230 to be advanced against cutter 400. When a certain pressure is achieved then cutter 400 cuts through target vessel 20.

FIGS. 7E-7F show just cutting assembly 745 of operator. However, it should be understood that compression assembly 760 shown in FIG. 6B is shown removed just to simply the illustration of movement of components in cutting assembly 745. Cutter 400 is moved back by the force of pulling anvil pull 230 which causes anvil 210 to be pushed against the vessels held within ring device 300 (not shown in FIGS. 7D-7F) and to transfer the force against cutting edge 414 of cutter 400. When cutter 400 is pushed back safety interlock 466 is also pushed back as safety interlock 466 is held in the cutter mount 458 of cutter 400. Spring mount 456 extending opposite from cutter mount 458 pushes against spring 460 until spring 460 has been compressed back a distance D, from its extended position within safety interlock space 468. Movement of safety interlock 466 causes the safety interlock indicator 467 to move back within slot 712 toward the distal end of operator 700. Movement of safety interlock 466 away from the anastomosis end of operator 700 enables safety interlock extension 468 to move back out of its locking position. As best seen in FIG. 7A and FIG. 7D, actuator knob 650 has a lock slot 652 which engages safety interlock extension 468 until safety interlock 466 has been moved. Safety interlock indicator 467 is positioned for the user to easily recognize that the cutter 400 has made an opening in the target vessel.

Once safety interlock extension 468 has been moved out of its locking position in lock slot 652 of actuator knob 650, then actuator knob 650 can be used to bring the rings together. This configuration prevents the premature compression of ring device 300.

As mentioned above, the upper portion of coupler 502 is referred to as an anvil pull holder portion. This portion and slot 532 are collectively referred to as anvil pull holder 530. As also mentioned above, the lower portion of coupler 502 is referred to as an anvil pull advancer portion. This portion, advancer or drive screw 562, threadably mounted advancer knob 570 with its contact post 578 bearing against bearing recess 572 are collectively referred to herein as an anvil pull advancer 560. These features work with cutter 400 and the spring assembly to control the pressure applied by anvil 210.

Anvil pull advancer 560 is adapted to pull anvil pull 230 once anvil pull 230 is held by anvil pull holder 530. As anvil pull advancer 560 pulls on anvil pull 230, it causes anvil 210 to advance within the anastomosis ring device and distend the wall of vessel 20 until cutter 400 is engaged by anvil 210 to cut through target vessel 20.

The anvil pull holder and the anvil pull advancer may be entirely separate components or have some common components such as coupler 502. Also the anvil pull holder and the anvil pull advancer may be embodied by a component capable of both holding and advancing the anvil pull. The anvil pull holder may also just lock the anvil pull into position such that the cutter is moved against a stationary anvil. Similarly, the spring biasing device 450 may be eliminated so that the vessel is cut only by pressure exerted by the anvil pull against the cutter.

The anvil pull holders disclosed herein are examples of holding means for holding the anvil pull extending from an anvil. The anvil pull advancers described herein are examples of advancement means for pulling the anvil pull once the anvil pull is held by the holding means. Anvil pull holder 530 and anvil pull advancer 560 are collectively referred to herein as anvil pull engager 500. Such an anvil pull engagers are examples of engaging means for holding an anvil pull extending from an anvil.

Actuator knob 650 has threads 654 (shown in FIG. 7D) at one end which are threadably coupled with body threads 648. Actuator knob 650 is rotatably mounted around compression advancer 600 opposite its threaded end. Rotation of actuator knob 650 causes the linear advancement of compression advancer 600. Compression advancer 600 is fixedly coupled to a slide tube 620 via a pin 644 which extends from compression advancer 600 to slide tube 620 via a groove 634 in tubular housing 640. This configuration enables actuator knob 650 to be rotated to urge compression advancer forward which then advances slide 620 against approximator 614.

Advancement of approximator 614 pushes approximator forward through ring retainer 610 against graft vessel ring 310b. This delivers an increase in pressure to ring device 300 as anvil pull 230 is held within slot 532. When sufficient force is applied, approximator 614 pushes the release prongs 350a of target vessel ring 310a out of retention receptacles 606 of ring retainer 610.

As shown in FIG. 6B, once the end-to-side anastomosis has been completed, then separator pin 642 is removed (it is shown after being reinserted) to separate tubular housing 640 from body 710 as shown in FIG. 6B. After tubular housing 640 of cutting assembly 760 and body 710 of compression assembly 745 have been separated then tubular housing 640 can be pulled over stented end 60 of graft vessel 50 to fully remove the operator 700 from graft vessel 50 as shown in FIG. 6C. The stented end 60 can then be attached to a vessel by a procedure such as the procedure illustrated in FIGS. 8A-8E.

FIGS. 8A-8E depict an exemplary set of steps for anastomosing the stented end of the graft vessel device to a vein following anastomosis of the other end to an artery. It should be understood that, although the vein anastomosis procedure is depicted as being done after the artery anastomosis procedure in the accompanying figures, this need not be the case. Some of the various steps in the vein anastomosis procedure may take place before or during any of the various steps in the artery anastomosis procedure. Similarly, other procedures are possible which require a reversal of the order disclosed herein.

In any event, if the artery anastomosis procedure has been accomplished first, the graft vessel device will typically be pinched, kinked, or otherwise occluded somewhere along its length during the subsequent vein anastomosis procedure to control blood flow. In FIG. 8A, this is shown being accomplished by the use of pincers 850.

To begin the vein anastomosis procedure, an introducer 802 and introducer wire 804 are inserted into a tear-away sheath 800. Tear-away sheath 800 may include gripping knobs 808 and a sheath portion 806. The tear-away sheath 800 with introducer 804 are inserted into an opening in the vein. The opening may be formed by a separate medical instrument or may be formed with introducer 802 and/or introducer wire 804.

Once the tear-away sheath 800 and introducer 802 have been inserted into the vein opening, as shown in FIG. 8A, sheath portion 806 of tear-away sheath 800 is clamped with pincers 850 or otherwise occluded to control blood flow, as shown in FIG. 8B. The stented end of the graft vessel device is then inserted into a second tear-away sheath 810. Optionally, the stented end of the graft vessel device may be folded, rolled, or otherwise compressed into the sheath portion 816 of the second tear-away sheath stent 360. In this manner, the stented end may be inserted into a sheath and/or vein having an equal or smaller diameter than that of the stented end of the graft vessel. FIG. 8C shows a cross-sectional view of one possible way in which the stent 50 and graft 360 could be folded within the sheath portion 816.

After the stented end of the graft vessel device has been inserted into tear-away sheath 810, tear-away sheath 810 is inserted into tear-away sheath 800 and into the opening in the vein. Once both tear-away sheaths and the stented end of the graft vessel device have been inserted into the vein, tear-away sheath 810 may then be pealed away and removed, as depicted in phantom in FIG. 8D. Tear-away sheath 800 may then be removed in the same manner, as also depicted in phantom in FIG. 8E. After both tear-away sheaths have been removed, the anastomosis procedure is complete. The graft vessel device at this stage with its stented end anastomosed to a vein and its other end anastomosed to artery 20 is depicted in FIG. 8F.

The stent may be attached to the graft vessel in any number of ways. It may, for instance, simply be sutured to the graft vessel. In other embodiments, the stent may be attached to the graft vessel by use of an adhesive such as a polymeric material. In such embodiments, the graft vessel may be placed over a mandrel. The stent may then be dipped into a solution containing one or more polymers. Examples of suitable polymers include polyurethane, polycarbonate urethanes (such as Bionate™, commercially available from The Polymer Technology Group of Berkeley, Calif.), silicones, polyethers, urethane copolymers, silicone urethane copolymers, polycarbonates, silicone polyether urethanes (such as PurSil™, also commercially available from The Polymer Technology Group), silicone polycarbonate (such as CarboSil™, also commercially available from The Polymer Technology Group), segmented polyurethane urethanes (such as BioSpan™, also commercially available from The Polymer Technology Group), polyetherurethanes (such as Elasthane™, also commercially available from The Polymer Technology Group), shape-memory thermoplastics (such as Calo-MER™, also commercially available from The Polymer Technology Group) siloxane polymers, Thermoplastic Polyurethanes (TPUs) (such as Tecoflex™, Tecothane™, Carbothane™, Tecophilic™, or Tecoplast™, each of which are commercially available from Noveon, Inc.) PTFE, ePTFE, etc.

Drug-eluting compositions may also be used to coat the stent or stented graft, such as is described in more detail in U.S. Pat. No. 5,591,227 titled “Drug Eluting Stents,” which was filed on Apr. 27, 1995, and which is hereby incorporated herein by reference in its entirety. Drug-eluting compositions may be used to prevent or control, for example, thrombosis and/or neointimal hyperplasia.

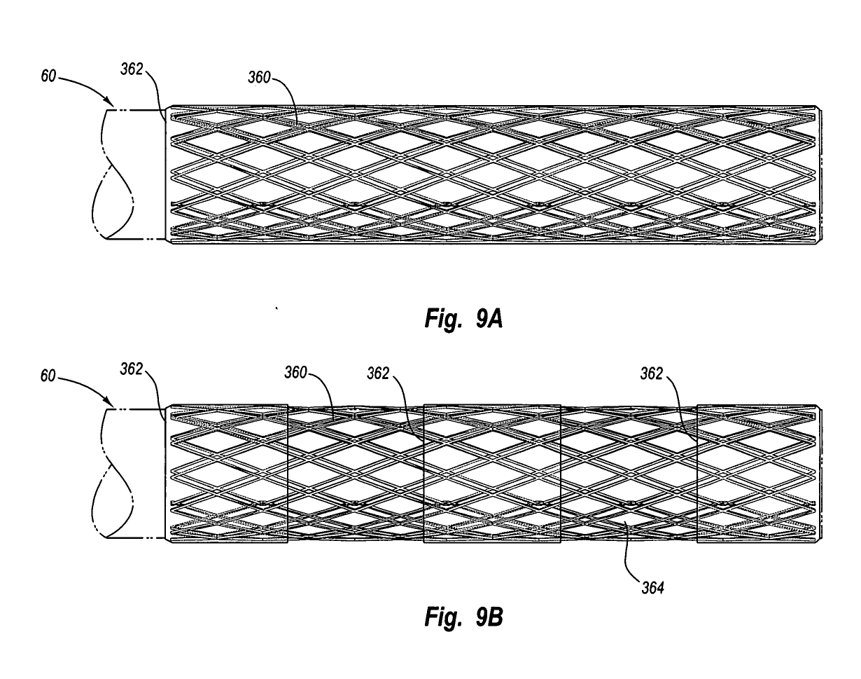

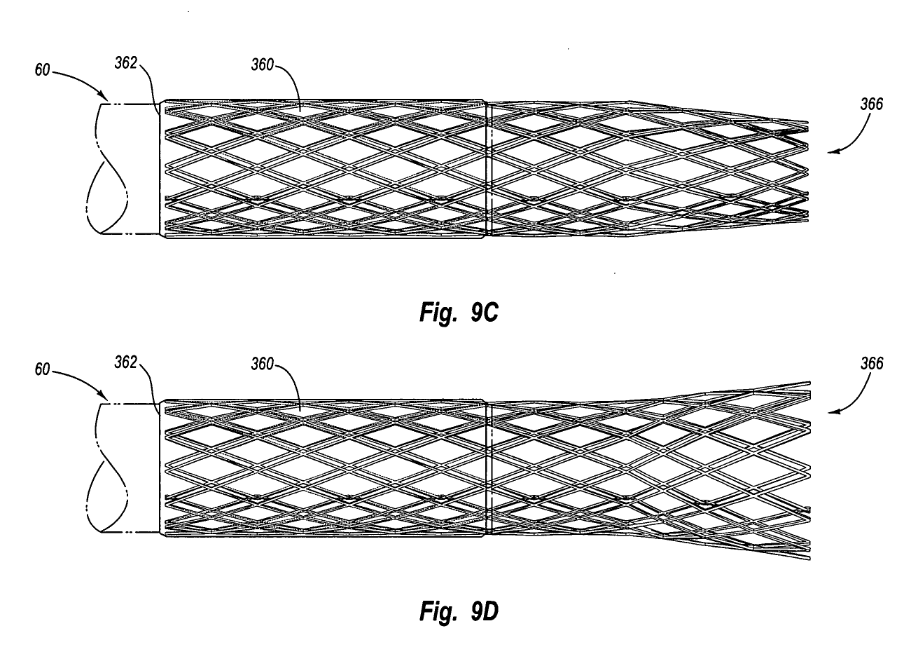

After being dipped in a suitable solution, the stent may then be placed over the graft vessel on the mandrel, after which it is allowed to dry, thereby fixedly attaching the stent to the graft vessel. If a curable polymer such as silicone is used, then the polymer is also cured at this time. It should be understood that the coating may also be applied to the stent/graft combination rather than just the stent. In other words, the stent may be attached or otherwise placed in or on the graft vessel first, after which the coating may be applied to the stent and graft vessel by dipping them into the coating substance or by any other suitable methodology discussed herein or otherwise available to one of skill in the art. The stented end of graft vessel 60 with coating 362 will then appear as in FIG. 9A. It should also be understood that the stent may instead be attached to the inside of the graft vessel. A second graft vessel may also be placed inside of the stent such that the stent is sandwiched between two graft vessels. Of course, the same objective may be achieved by attaching the stent to the outside surface of the graft vessel, as discussed above, and then fitting a second graft vessel over the stented graft.

A coating may also be formed by impregnating a porous graft vessel with a polymer. The polymer becomes integrated in the graft interstices, resulting in a coating which has substantially lower permeability than the original graft vessel and secures the stent onto its surface.

A coating may also be formed by providing a stent-graft having a stent on the inside layer and a braided PET graft on the outside layer. The stent-graft may be placed over a mandrel which has an outer diameter similar to the inner diameter of the stent-graft. The ends of the mandrel may then be affixed in a machine which rotates the stent-graft and mandrel about a central axis. Using an airbrush or similar spraying apparatus, the stent-graft may be sprayed with a solution of silicone or other suitable polymer in a volatile solvent such as tetrahydrofuran. A suitable volume, possibly around 10 cc, of silicone solution may be sprayed intermittently over a period of time onto the stent-graft. The coated stent-graft and mandrel may then be placed in an oven to cure the silicone polymer. An additional graft layer may be bonded on the inside or outside of the graft vessel device depending on biocompatibility needs. For example, if a silicone has been embedded in the graft vessel, the silicone may extend to the inside surface where blood flow will occur. In that case, it may be desirable to add a PET braided graft vessel on the inside for biocompatibility purposes.

As an additional possible method for attaching the stent to the graft vessel, a polymeric film or tube may be applied to the inside and/or outside surface of the stent or stent-graft. The film and/or tube may be fused to the stent or stent-graft by use of adhesive, solvent bonding, or by thermal and/or pressure bonding.

Other suitable methods for attaching a stent to a graft vessel can be found in U.S. Pat. No. 6,709,455 titled “Stent-Graft-Membrane and Method of Making the Same,” which was filed on Oct. 10, 2000. The disclosure of this patent is hereby incorporated herein by reference in its entirety.

Coating 362 may alternatively be segmented at the stented end of graft vessel 60, as shown in FIG. 9B. By segmenting the coating 362, the stent may be reliably attached to the graft vessel at the coated segments, and may also be allowed to over time be incorporated into the cellular matrix of a blood vessel it is inserted into at the uncoated segments. Creating such a segmented coating may be accomplished by masking one or more segments of the stent prior to dipping the stent or otherwise applying the coating.

It should be understood that stent 360 may flare inward or outward at one end, as depicted in FIGS. 9C and 9D, respectively. Flaring an end of the stent 360 will allow for customization of anastomotic properties and use of the device on vessels of varying diameters. The flared portion of the stent may be coated, uncoated, or partially coated as desired. Also as depicted in FIGS. 9C and 9D, any of the embodiments discussed herein may have one half or a single portion of the stent 360 coated. Most typically, the end of the stent that is to be inserted into a blood vessel will be the end that is not coated, as also depicted in FIGS. 9C and 9D.

It will be obvious to those having skill in the art that many changes may be made to the details of the above-described embodiments without departing from the underlying principles of the invention. The scope of the present invention should, therefore, be determined only by the following claims. Note also that elements recited in means-plus-function format are intended to be construed in accordance with 35 U.S.C. § 112 ¶6.

1. A method for connecting a vessel to another vessel comprising:
obtaining a graft vessel device comprising a graft vessel, an anastomosis device attached to a first end of the graft vessel and a stent attached to a second end of the graft vessel,
anastomosing the first end of the graft vessel to a side of a first vessel via the anastomosis device to yield an end-to-side anastomosis, and
anastomosing the second end of the graft vessel to a second vessel via the stent to yield an end-to-end anastomosis.

2. The method of claim 1, wherein the anastomosis device comprises two rings.

3. The method of claim 1, wherein the anastomosis device comprises a first anastomosis ring adapted to cooperate with a second anastomosis ring.

4. The method of claim 1, wherein the stent is fixedly attached to the exterior surface of the graft vessel at the second end.

5. The method of claim 1, wherein the second end of the graft vessel is attached to the second vessel by inserting the second end of the graft vessel into the lumen of the second vessel.

6. The method of claim 1, wherein the second end of the graft vessel is attached to the second vessel by inserting the second end of the graft vessel into the lumen of the second vessel via at least one tear-away sheath.

7. The method of claim 1, wherein the second end of the graft vessel is attached to the second vessel by inserting the second end of the graft vessel into the lumen of the second vessel and then allowing the stent to unfold.

8. The method of claim 1, wherein the second end of the graft vessel is attached to the second vessel by inserting the second end of the graft vessel into the lumen of the second vessel and then allowing the expand.

9. The method of claim 1, wherein the first end of the graft vessel is attached to the first vessel and then the second end of the graft vessel is attached to the second vessel.

10. The method of claim 1, further comprising loading the graft vessel device into a tubular housing of an operator used to attach the first end of the graft vessel to the side of the first vessel in cooperation with the anastomosis device to yield the end-to-side anastomosis.

11. The method of claim 1, further comprising:
loading the graft vessel device into a tubular housing of an operator used to attach the first end of the graft vessel to the side of the first vessel in cooperation with the anastomosis device to yield the end-to-side anastomosis, and
removing the graft vessel device from the tubular housing of the operator to attach the second end of the graft vessel to the second vessel to yield the end-to-end anastomosis.

12. A graft vessel device comprising:
a graft vessel;
an anastomosis device attached to a first end of the graft vessel,
wherein the anastomosis device is configured to facilitate end-to-side anastomosis of the graft vessel device to a side of a first vessel; and
a stent attached to a second end of the graft vessel.

13. A graft vessel device of claim 12, wherein the stent is configured to facilitate anastomosis of the second end of the graft vessel to a second vessel to yield an end-to-end anastomosis.

14. A graft vessel device of claim 12, wherein the stent is attached to the exterior surface of the graft vessel at the second end of the graft vessel device.

15. The graft vessel device of claim 12, wherein the stent is sutured to the graft vessel at the first end of the graft vessel device.

16. The graft vessel device of claim 12, wherein the stent is fixedly attached to the graft vessel with an adhesive.

17. The graft vessel device of claim 12, wherein the stent is fixedly attached to the graft vessel with a polymeric substance.

18. The graft vessel device of claim 12, wherein the stent is fixedly attached to the graft vessel with polyurethane.

19. A graft vessel device comprising:
a graft vessel having a first end opposite from a second end;
an anastomosis ring attached to the first end of the graft vessel,
wherein the anastomosis ring is configured to facilitate end-to-side anastomosis of the graft vessel device to a first vessel via cooperation with another anastomosis ring; and
a stent attached to the second end of the graft vessel.

20. A graft vessel device of claim 19, wherein the stent is configured to facilitate anastomosis of the second end of the graft vessel to a second vessel to yield an end-to-end anastomosis.

21. The graft vessel device of claim 19, wherein the stent is attached to the exterior surface of the graft vessel at the first end of the graft vessel device.

22. The graft vessel device of claim 19, wherein the stent is sutured to the graft vessel at the first end of the graft vessel device.

23. The graft vessel device of claim 19, wherein the stent is fixedly attached to the graft vessel with an adhesive.

24. The graft vessel device of claim 19, wherein the stent is fixedly attached to the graft vessel with a polymeric substance.

25. The graft vessel device of claim 19, wherein the stent is fixedly attached to the graft vessel with polyurethane.
